SEQUENCE LISTING (23 sequences)

SEQ ID NO: 1 - protein, 7 aa, synthetic construct
SGSYFWG

SEQ ID NO: 2 - protein, 16 aa, synthetic construct
SIYYSGITYYNPSLKS

SEQ ID NO: 3 - protein, 11 aa, synthetic construct
HDGAVAGLFDY

SEQ ID NO: 4 - protein, 11 aa, synthetic construct
GGNNIGSKSVH

SEQ ID NO: 5 - protein, 7 aa, synthetic construct
DDSDRPS

SEQ ID NO: 6 - protein, 11 aa, synthetic construct
QVWDSSSDHVV

SEQ ID NO: 7 - protein, 121 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 8 - protein, 108 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 9 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 11 - protein, 5 aa, synthetic construct
TYAMN

SEQ ID NO: 12 - protein, 19 aa, synthetic construct
RIRSKYNNYATYYAASVKG

SEQ ID NO: 13 - protein, 14 aa, synthetic construct
HGNFGNSYVSWFAY

SEQ ID NO: 14 - protein, 14 aa, synthetic construct
RSSTGAVTTSNYAN

SEQ ID NO: 15 - protein, 7 aa, synthetic construct
GTNKRAP

SEQ ID NO: 16 - protein, 9 aa, synthetic construct
ALWYSNLWV

SEQ ID NO: 17 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 18 - protein, 112 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 19 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 20 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 22 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 23 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

(19) United States
(12) Patent Application Publication
(10) Pub. No.: US 2024/0400702 A1
(43) Pub. Date: Dec. 5, 2024

(54) PHARMACEUTICAL COMPOSITIONS COMPRISING A BISPECIFIC BCMA/CD3 ANTIBODY AT HIGH CONCENTRATION

(71) Applicant: Janssen Biotech, Inc., Horsham, PA (US)

(72) Inventors: Michael Zakrewsky, Philadelphia, PA (US); Shyamal Choudhari, Malvern, PA (US)

(21) Appl. No.: 18/657,851

(22) Filed: May 8, 2024

Related U.S. Application Data
(60) Provisional application No. 63/465,016, filed on May 9, 2023.

(51) Int. Cl. C07K 16/28

(52) U.S. Cl. CPC C07K 16/2878; C07K 16/2809; C07K 2317/24; C07K 2317/31

(57) ABSTRACT

Provided herein are aqueous pharmaceutical compositions comprising high-concentration formulations of a bispecific BCMA/CD3 antibody or an antigen-binding fragment thereof, and methods of preparing the same. Also provided herein are methods of treating cancer in a subject in need thereof by administering to the subject the aqueous pharmaceutical compositions.

### CROSS-REFERENCE TO RELATED APPLICATIONS

This application claims priority to U.S. Provisional Application Ser. No. 63/465,016, filed May 9, 2023, the entire contents of which are incorporated herein by reference in their entirety.

### REFERENCE TO SEQUENCE LISTING SUBMITTED ELECTRONICALLY

This application contains a sequence listing, which is submitted electronically in XML format and is hereby incorporated by reference in its entirety. Said XML copy, created on Apr. 30, 2024, is named “258199062101 (JBI6808USNP1) Sequence Listing.xml” and is 23,201 bytes in size.

### FIELD OF THE INVENTION

Disclosed herein are compositions and methods for formulating a stable pharmaceutical composition comprising bispecific BCMA/CD3 antibodies at high concentration.

### BACKGROUND OF THE INVENTION

Multiple myeloma (MM) is a cancer of the plasma cells. Mechanistically, multiple myeloma is characterized by production of monoclonal proteins (M-proteins) comprised of pathological immunoglobulins or fragments of such, which have lost their function. The proliferation of multiple myeloma cells leads to subsequent displacement from the normal bone marrow niche, while overproduction of M-proteins causes characteristic osteolytic lesions, increased susceptibility to infections, hypercalcemia, renal insufficiency or failure, and neurological complications.

Treatment options for multiple myeloma have improved over time and vary depending on the aggressiveness of the disease, underlying prognostic factors, physical condition of the patient, and existing comorbidities. Therapeutic options include proteasome inhibitors (PIs), immunomodulatory drugs (IMiDs), alkylating agents, monoclonal antibodies (mAbs), antibody drug conjugate, histone deacetylase inhibitor, nuclear protein export inhibitor, chimeric antigen receptor (CAR) T cell therapy and stem cell transplantation.

Despite these therapeutic achievements, the disease recurs and is associated with additional risk factors (e.g., comorbidities or increasing age), thus warranting the need for novel therapeutic approaches, such as new drug product formulations, and new dosage and treatment regimens. In particular in the elderly population, for which stem cell transplantation is often not a viable option, and in patients with refractory disease who exhausted several therapies, multiple myeloma remains an incurable malignancy and an unmet medical need with significant morbidity and mortality.

While anti-BCMA/CD3 bispecific antibodies have shown promising results, there remains a need in the art for pharmaceutical compositions comprising such antibodies that are stable for long periods of time at refrigerated (2-8° C.) and ambient temperatures. There also remains a need in the art for pharmaceutical compositions comprising high concentrations of such antibodies while still having a suitable viscosity, e.g., for subcutaneous administration.

### SUMMARY OF THE INVENTION

Disclosed herein are aqueous pharmaceutical compositions comprising specific formulations of a bispecific B-cell mature antigen (BCMA)/cluster of differentiation 3 (CD3) antibody. In certain embodiments, the compositions have a high concentration of the bispecific antibody, e.g., 150 mg/mL or greater, while also having a low viscosity, e.g., 25 centipoise (cP) or less, and a stable shelf life. A pharmaceutical composition having a high concentration of a therapeutic antibody enables patients to receive fewer subcutaneous injections to achieve a therapeutic dose than would be required with a composition having a lower concentration of antibody, thus reducing the risk of complications such as injection site reactions and improving the patient's quality of life. The inventors have developed high-concentration compositions that are stable over time while having viscosities that are low enough to be suitable for subcutaneous injection.

In one general aspect, provided herein are aqueous pharmaceutical compositions comprising:
a) about 150 mg/mL to about 250 mg/mL of a bispecific B-cell mature antigen (BCMA)/cluster of differentiation 3 (CD3) antibody, the bispecific BCMA/CD3 antibody comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively; and
b) a viscosity reducing agent (e.g., arginine HCl), wherein the composition has a viscosity of no more than about 25 centipoise (cP) at 25° C. In embodiments described herein, the pharmaceutical composition is stable.

In another aspect, provided herein are aqueous pharmaceutical compositions comprising:
(a) about 100 mg/mL to about 300 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 1% (w/v) to about 7% (w/v) of sucrose;
(d) about 100 mM to about 300 mM arginine HCl;
(e) about 16 μg/mL to about 24 μg/mL of ethylenediaminetetraacetic acid (EDTA); and
(f) about 0.01% to about 0.07% polysorbate 20;
wherein the composition has a pH from about 4.7 to about 5.7. In embodiments described herein, the pharmaceutical composition is stable.

In another general aspect, provided herein are aqueous pharmaceutical compositions comprising:
(a) about 175 mg/mL to about 225 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 2% (w/v) to about 6% (w/v) of sucrose;
(d) about 175 mM to about 225 mM arginine HCl;
(e) about 16 μg/mL to about 24 μg/mL of ethylenediaminetetraacetic acid (EDTA);
(f) about 0.01% to about 0.07% polysorbate 20; and
a pH from about 4.7 to about 5.7. In embodiments described herein, the pharmaceutical composition is stable.

In another aspect, provided herein are aqueous pharmaceutical compositions comprising:
(a) about 7.5 mg/mL to about 12.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 6% (w/v) to about 10% (w/v) of sucrose;
(d) about 6 mM to about 14 mM arginine HCl;
(e) about 16 μg/mL to about 24 μg/mL of ethylenediaminetetraacetic acid (EDTA);
(f) about 0.01% to about 0.07% polysorbate 20; and
(g) a pH from about 4.7 to about 5.7. In embodiments described herein, the pharmaceutical composition is stable.

According to preferred embodiments the aqueous pharmaceutical compositions described herein are stable aqueous pharmaceutical compositions.

Also provided herein are methods of treating cancer in a subject in need thereof. The methods comprise administering to the subject the aqueous pharmaceutical compositions, as disclosed herein.

### DETAILED DESCRIPTION OF THE INVENTION

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

The term “comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The term “antibody,” and like terms is meant in a broad sense and includes immunoglobulin molecules or fragments thereof, including monoclonal antibodies (such as murine, human, human-adapted, humanized, and chimeric monoclonal antibodies), antibody fragments, bispecific or multispecific antibodies, dimeric, tetrameric or multimeric antibodies, and single chain antibodies.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antibody fragment” refers to a portion of an immunoglobulin molecule that retains the antigen binding properties of the parental full-length antibody. Exemplary antibody fragments are heavy chain complementarity determining regions (HCDR) 1, 2, and 3, light chain complementarity determining regions (LCDR) 1, 2, and 3, a heavy chain variable region (VH), or a light chain variable region (VL). Antibody fragments include: a Fab fragment, a monovalent fragment consisting of the VL, VH, constant light (CL), and constant heavy 1 (CH1) domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; and a domain antibody (dAb) fragment (Ward et al., Nature 341:544-546, 1989), which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Int'l Pat. Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

An antibody variable region consists of a “framework” region interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132:211-50, 1970; Kabat et al. Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Monoclonal antibody” refers to a preparation of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Bispecific” refers to an antibody that specifically binds two distinct antigens or two distinct epitopes within the same antigen. The bispecific antibody can have cross-reactivity to other related antigens, for example to the same antigen from other species (homologs), such as human or monkey, for example Macaca cynomolgus (cynomolgus, cyno) or Pan troglodytes, or can bind an epitope that is shared between two or more distinct antigens.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody can include substitutions in the frameworks so that the frameworks cannot be exact copies of expressed human immunoglobulin or human immunoglobulin germline gene sequences.

“Identity” refers to a relationship between the sequences of two or more polypeptide molecules or two or more nucleic acid molecules, as determined by aligning and comparing the sequences. “Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“BCMA/CD3 bispecific antibody” (which may be used interchangeably with “BCMAxCD3 bispecific antibody”) refers to a bispecific antibody that specifically binds BCMA and CD3. BCMA/CD3 bispecific antibodies are described in U.S. Pat. No. 10,072,088, which is incorporated by reference herein in its entirety.

“BCMA” refers to human B-cell maturation antigen, also known as CD269 or TNFRSF17 (UniProt Q02223). The extracellular domain of BCMA encompasses residues 1-54 of Q02223. Human BCMA comprises the amino acid sequence of SEQ ID NO:21.

(SEQ ID NO: 21)
MLQMAGQCSQNEYFDSLLHACIPCQLRCSSNTPPLTCQRYCNASV
TNSVKGTNAILWTCLGLSLIISLAVFVLMFLLRKINSEPLKDEFK
NTGSGLLGMANIDLEKSRTGDEIILPRGLEYTVEECTCEDCIKSK
PKVDSDHCFPLPAMEEGATILVTTKTNDYCKSLPAALSATEIEKS
ISAR

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO:22. SEQ ID NO:23 shows the extracellular domain of CD3 epsilon.

(SEQ ID NO: 22)
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRQRGQNKERP
PPVPNPDYEPIRKGQRDLYSGLNQRRI
(SEQ ID NO: 23)
DGNEEMGGITQTPYKVSISGTTVILTCPQYPGSEILWOHNDKNIG
GDEDDKNIGSDEDHLSLKEFSELEQSGYYVCYPRGSKPEDANFYL
YLRARVCENCMEMD

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions, or deletions.

“In combination with” means that two or more therapeutics can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, improving or remediating damage caused, directly or indirectly, by the malignancy. Treatment also includes prolonging survival as compared to the expected survival of a subject not receiving treatment. Subjects to be treated include those that have the condition or disorder as well as those prone to have the condition or disorder or those in which the condition or disorder is to be prevented.

“Therapeutically effective amount” refers to an amount of the disclosed com position, which is therapeutically effective at dosages and for periods of time necessary, to achieve a desired treatment. A therapeutically effective amount may vary according to factors such as the disease state, age, sex, and weight of the subject, and the ability of the combination therapy to elicit a desired response in the subject. Exemplary indicators of a therapeutically effect amount include, for example, improved well-being of the patient, reduction of a tumor burden, arrested or slowed growth of a tumor, and/or absence of metastasis of cancer cells to other locations in the body.

“Pharmaceutical composition” refers to composition that comprises an active ingredient and a pharmaceutically acceptable carrier.

The term “drug product” or “DP” may be used interchangeably herein with the term “aqueous pharmaceutical composition.”

“Pharmaceutically acceptable carrier” or “excipient” refers to an ingredient in a pharmaceutical composition, other than the active ingredient, which is nontoxic to a subject.

The term “cancer” as used herein is defined as disease characterized by the rapid and uncontrolled growth of aberrant cells. Cancer cells can spread locally or through the bloodstream and lymphatic system to other parts of the body. In certain embodiments, the cancer is a hematological malignancy or a solid tumor. In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“T cell redirecting therapeutic” refers to a molecule containing two or more binding regions, wherein one of the binding regions specifically binds a cell surface antigen on a target cell or tissue and wherein a second binding region of the molecule specifically binds a T cell antigen. Examples of cell surface antigen include a tumor associated antigen, such as BCMA. Examples of T cell antigen include, e.g., CD3. This dual/multi-target binding ability recruits T cells to the target cell or tissue leading to the eradication of the target cell or tissue.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

As used herein, “stable” or “stability” refers to the pharmaceutical composition and the extent to which the bispecific antibody, or antigen binding fragment thereof, retains, within specified limits and throughout its period of storage and use, the same or nearly the same properties and characteristics that is possessed at the time of its manufacture. These antibody, or antigen binding fragment thereof, properties and characteristics can be, for example, activity, aggregation or lack thereof, degradation products or lack thereof, or other relevant measurements as described herein.

### Description

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Compositions comprising BCMAxCD3 bispecific antibodies

Disclosed herein are aqueous pharmaceutical compositions comprising a bispecific BCMA/CD3 antibody. In particular embodiments, the pharmaceutical composition is stable. In certain embodiments, the compositions have a high concentration of the bispecific antibody, e.g., 150 mg/mL or greater, while also having a low viscosity, e.g., 25 centipoise (cP) or less. The inventors have discovered that compositions comprising a higher concentration of a bispecific BCMA/CD3 antibody can be formulated to be stable and to have a low enough viscosity so they are suitable for subcutaneous administration to human subjects. A pharmaceutical composition having a high concentration of a therapeutic antibody enables patients to receive fewer subcutaneous injections, than would be required with a composition having a lower concentration of antibody, to achieve a therapeutic dose, thus reducing the risk of complications such as injection site reactions and improving the patient's quality of life.

Embodiments of the present invention provide aqueous pharmaceutical compositions having a lower concentration of antibody which may be particularly useful as step-doses in a treatment regimen, as well as pharmaceutical concentrations having a high concentration of antibody which may be particularly useful as treatment doses in a treatment regimen. As used herein, a “step-up dose” refers to a dose of an active agent that is administered to a subject prior to a treatment dose. A step-up dose is lower than the treatment dose. To prevent or lessen certain toxicities, such as cytokine release syndrome (CRS), a “priming” dose strategy may include one or more lower step-up dose(s) followed by higher treatment doses. As used herein, a “treatment dose” refers to a dose of the active agent that is administered to a subject to treat a disease. A treatment dose may be administered at a regular dosing interval on a repetitive basis (e.g. weekly). A treatment dose may be preceded by one or more step-up doses.

In one general aspect, provided herein are aqueous pharmaceutical compositions comprising:
a) about 150 mg/mL to about 250 mg/mL of a bispecific B-cell mature antigen (BCMA)/cluster of differentiation 3 (CD3) antibody, the bispecific BCMA/CD3 antibody comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively; and
b) a viscosity reducing agent (e.g., arginine HCl), wherein the composition has a viscosity of no more than about 25 centipoise (cP) at 25° C. Such high-concentration compositions may be particularly useful as treatment doses. In embodiments described herein, the pharmaceutical composition is stable.

In another aspect, provided herein are aqueous pharmaceutical compositions comprising:
(a) about 7.5 mg/mL to about 12.5 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate
(c) about 6% (w/v) to about 10% (w/v) of sucrose;
(d) about 6 mM to about 14 mM arginine HCl;
(e) about 16 μg/mL to about 24 μg/mL of ethylenediaminetetraacetic acid (EDTA); and
(f) about 0.01% to about 0.07% polysorbate 20;
   wherein the composition has a pH from about 4.7 to about 5.7. Such compositions may be particularly useful as step-up doses. In embodiments described herein, the pharmaceutical compositions are stable.

In another general aspect, provided herein are aqueous pharmaceutical compositions comprising:
(a) about 100 mg/mL to about 300 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate
(c) about 1% (w/v) to about 7% (w/v) of sucrose;
(d) about 100 mM to about 300 mM arginine HCl;
(e) about 16 μg/mL to about 24 μg/mL of ethylenediaminetetraacetic acid (EDTA); and
(f) about 0.01% to about 0.07% polysorbate 20;
   wherein the composition has a pH from about 4.7 to about 5.7. Such high-concentration compositions are particularly useful as treatment doses. In embodiments described herein, the pharmaceutical composition is stable.

In another general aspect, provided herein are aqueous pharmaceutical compositions comprising:
(a) about 175 mg/mL to about 225 mg/mL of a bispecific B-cell mature antigen (BCMA)/cluster of differentiation 3 (CD3) antibody or antigen-binding fragment thereof, the bispecific BCMA/CD3 antibody or antigen-binding fragment thereof comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 2% (w/v) to about 6% (w/v) of sucrose;
(d) about 175 mM to about 225 mM arginine HCl;
(e) about 16 μg/mL to about 24 μg/mL of ethylenediaminetetraacetic acid (EDTA); and
(f) about 0.01% to about 0.07% polysorbate 20;
   wherein the composition has a pH from about 4.7 to about 5.7. Such high-concentration compositions are particularly useful for treatment doses.

According to preferred embodiments the aqueous pharmaceutical compositions described herein are stable aqueous pharmaceutical compositions.

In certain embodiments, the bispecific BCMA/CD3 antibody comprises a VH1 having the amino acid sequence of SEQ ID NO:7 and a VL1 having the amino acid sequence of SEQ ID NO:8. In certain embodiments, the bispecific BCMA/CD3 antibody comprises a HC1 having the amino acid sequence of SEQ ID NO:9, and a LC1 having the amino acid sequence of SEQ ID NO:10. In certain embodiments, the bispecific BCMA/CD3 antibody comprises a HC1 having at least 90%, at least 95%, at least 98%, or at least 99% sequence identity to the amino acid sequence of SEQ ID NO:9. In certain embodiments, the bispecific BCMA/CD3 antibody comprises a LC1 having at least 90%, at least 95%, at least 98%, or at least 99% sequence identity to the amino acid sequence of SEQ ID NO:10.

In certain embodiments, the bispecific BCMA/CD3 antibody comprises a VH2 having the amino acid sequence of SEQ ID NO:17, and a VL2 having the amino acid sequence of SEQ ID NO:18. In certain embodiments, the bispecific BCMA/CD3 antibody comprises a HC2 having the amino acid sequence of SEQ ID NO:19, and a LC2 having the amino acid sequence of SEQ ID NO:20. In certain embodiments, the bispecific BCMA/CD3 antibody comprises a HC2 having at least 90%, at least 95%, at least 98%, or at least 99% sequence identity to the amino acid sequence of SEQ ID NO:19. In certain embodiments, the bispecific BCMA/CD3 antibody comprises a LC2 having at least 90%, at least 95%, at least 98%, or at least 99% sequence identity to the amino acid sequence of SEQ ID NO:20.

Tables 2 and 3 provide sequences of an exemplary embodiment of a BCMA/CD3 bispecific antibody, according to the Kabat numbering system.

TABLE 2
Exemplary sequences of a BCMA binding arm

| | | | SEQ ID |
|---|---|---|---|
| | Region | Sequence | NO: |
| BCMA | HCDR1 | SGSYFWG | 1 |
| Binding | | | |
| Arm | | | |
| BCMB69 | | | |
| | HCDR2 | SIYYSGITYYNPSLKS | 2 |
| | HCDR3 | HDGAVAGLFDY | 3 |
| | LCDR1 | GGNNIGSKSVH | 4 |
| | LCDR2 | DDSDRPS | 5 |
| | LCDR3 | QVWDSSSDHVV | 6 |
| | VH1 | QLQLQESGPGLVKPSETLSL | 7 |
| | | TCTVSGGSISSGSYFWGWIR | |
| | | QPPGKGLEWIGSIYYSGITY | |
| | | YNPSLKSRVTISVDTSKNQF | |
| | | SLKLSSVTAADTAVYYCARH | |
| | | DGAVAGLFDYWGQGTLVTVS | |
| | | S | |
| | VL1 | SYVLTQPPSVSVAPGQTARI | 8 |
| | | TCGGNNIGSKSVHWYQQPPG | |
| | | QAPVVVVYDDSDRPSGIPER | |
| | | FSGSNSGNTATLTISRVEAG | |
| | | DEAVYYCQVWDSSSDHVVFG | |
| | | GGTKLTVL | |
| | HC1 | QLQLQESGPGLVKPSETLSL | 9 |
| | | TCTVSGGSISSGSYFWGWIR | |
| | | QPPGKGLEWIGSIYYSGITY | |
| | | YNPSLKSRVTISVDTSKNQF | |
| | | SLKLSSVTAADTAVYYCARH | |
| | | DGAVAGLFDYWGQGTLVTVS | |
| | | SASTKGPSVFPLAPCSRSTS | |
| | | ESTAALGCLVKDYFPEPVTV | |
| | | SWNSGALTSGVHTFPAVLQS | |
| | | SGLYSLSSVVTVPSSSLGTK | |
| | | TYTCNVDHKPSNTKVDKRVE | |
| | | SKYGPPCPPCPAPEAAGGPS | |
| | | VFLFPPKPKDTLMISRTPEV | |
| | | TCVVVDVSQEDPEVQFNWYV | |
| | | DGVEVHNAKTKPREEQFNST | |
| | | YRVVSVLTVLHQDWLNGKEY | |
| | | KCKVSNKGLPSSIEKTISKA | |
| | | KGQPREPQVYTLPPSQEEMT | |
| | | KNQVSLTCLVKGFYPSDIAV | |
| | | EWESNGQPENNYKTTPPVLD | |
| | | SDGSFFLYSRLTVDKSRWQE | |
| | | GNVFSCSVMHEALHNHYTQK | |
| | | SLSLSLGK | |
| | LC1 | SYVLTQPPSVSVAPGQTARI | 10 |
| | | TCGGNNIGSKSVHWYQQPPG | |
| | | QAPVVVVYDDSDRPSGIPER | |
| | | FSGSNSGNTATLTISRVEAG | |
| | | DEAVYYCQVWDSSSDHVVFG | |
| | | GGTKLTVLGQPKAAPSVTLF | |
| | | PPSSEELQANKATLVCLISD | |
| | | FYPGAVTVAWKGDSSPVKAG | |
| | | VETTTPSKQSNNKYAASSYL | |
| | | SLTPEQWKSHRSYSCQVTHE | |
| | | GSTVEKTVAPTECS | |

TABLE 3
Exemplary sequences of a CD3 binding arm

| | | | SEQ |
|---|---|---|---|
| | Region | Sequence | ID NO: |
| CD3 | HCDR1 | TYAMN | 11 |
| Binding | | | |
| Arm | | | |
| CD3B219 | | | |
| | HCDR2 | RIRSKYNNYATYYAASVKG | 12 |
| | HCDR3 | HGNFGNSYVSWFAY | 13 |
| | LCDR1 | RSSTGAVTTSNYAN | 14 |
| | LCDR2 | GTNKRAP | 15 |
| | LCDR3 | ALWYSNLWV | 16 |
| | VH2 | EVQLVESGGGLVQPGGSLRL | 17 |
| | | SCAASGFTFNTYAMNWVRQA | |
| | | PGKGLEWVARIRSKYNNYAT | |
| | | YYAASVKGRFTISRDDSKNS | |
| | | LYLQMNSLKTEDTAVYYCAR | |
| | | HGNFGNSYVSWFAYWGQGTL | |
| | | VTVSS | |
| | VL2 | QTVVTQEPSLTVSPGGTVTL | 18 |
| | | TCRSSTGAVTTSNYANWVQQ | |
| | | KPGQAPRGLIGGTNKRAPGT | |
| | | PARFSGSLLGGKAALTLSGV | |
| | | QPEDEAEYYCALWYSNLWVF | |
| | | GGGTKLTVLGQP | |
| | HC2 | EVQLVESGGGLVQPGGSLRL | 19 |
| | | SCAASGFTENTYAMNWVRQA | |
| | | PGKGLEWVARIRSKYNNYAT | |
| | | YYAASVKGRFTISRDDSKNS | |
| | | LYLQMNSLKTEDTAVYYCAR | |
| | | HGNFGNSYVSWFAYWGQGTL | |
| | | VTVSSASTKGPSVFPLAPCS | |
| | | RSTSESTAALGCLVKDYFPE | |
| | | PVTVSWNSGALTSGVHTFPA | |
| | | VLQSSGLYSLSSVVTVPSSS | |
| | | LGTKTYTCNVDHKPSNTKVD | |
| | | KRVESKYGPPCPPCPAPEAA | |
| | | GGPSVFLFPPKPKDTLMISR | |
| | | TPEVTCVVVDVSQEDPEVQF | |
| | | NWYVDGVEVHNAKTKPREEQ | |
| | | FNSTYRVVSVLTVLHQDWLN | |
| | | GKEYKCKVSNKGLPSSIEKT | |
| | | ISKAKGQPREPQVYTLPPSQ | |
| | | EEMTKNQVSLTCLVKGFYPS | |
| | | DIAVEWESNGQPENNYKTT | |
| | | PPVLDSDGSFLLYSKLTVDK | |
| | | SRWQEGNVFSCSVMHEALHN | |
| | | HYTQKSLSLSLGK | |
| | LC2 | QTVVTQEPSLTVSPGGTVTL | 20 |
| | | TCRSSTGAVTTSNYANWVQQ | |
| | | KPGQAPRGLIGGTNKRAPGT | |
| | | PARFSGSLLGGKAALTLSGV | |
| | | QPEDEAEYYCALWYSNLWVF | |
| | | GGGTKLTVLGQPKAAPSVTL | |
| | | FPPSSEELQANKATLVCLIS | |
| | | DFYPGAVTVAWKADSSPVKA | |
| | | GVETTTPSKQSNNKYAASSY | |
| | | LSLTPEQWKSHRSYSCQVTH | |
| | | EGSTVEKTVAPTECS | |

The bispecific BCMA/CD3 antibody can, for example, be teclistamab.

### Low-Concentration Aqueous Pharmaceutical Compositions

In certain embodiments, the bispecific BCMA/CD3 antibody has a concentration that is especially suitable for step-up doses, e.g., a concentration of about 7 mg/mL to about 13 mg/mL, about 7.5 mg/mL to about 12.5 mg/mL, about 8 mg/mL to about 12 mg/mL, or about 9 mg/mL to about 11 mg/mL. The bispecific BCMA/CD3 antibody can, for example, have a concentration of about 7 mg/mL, about 7.5 mg/mL, about 8 mg/mL, about 9 mg/mL, about 10 mg/mL, about 11 mg/mL, about 12 mg/mL, about 12.5 mg/mL, or about 13 mg/mL, or any value in between. In particular embodiments, the bispecific BCMA/CD3 antibody has a concentration of about 10 mg/mL.

In certain embodiments, the composition comprises about 5 mM Arginine HCl to about 15 mM Arginine HCl, or about 6 mM Arginine HCl to about 14 mM Arginine HCl, or about 7 mM Arginine HCl to about 13 mM Arginine HCl or about 8 mM Arginine HCl to about 12 mM Arginine HCl, or about 9 mM Arginine HCl to about 11 mM Arginine HCl, or any value in between. In a particular embodiment, the composition comprises about 10 mM Arginine HCl.

In certain embodiments, the composition comprises about 10 mM to about 20 mM, about 12 mM to about 18 mM, or about 14 mM to about 16 mM of acetate and/or a pharmaceutically acceptable acetate salt. The composition can, for example, comprise about 10 mM, about 11 mM, about 12 mM, about 13 mM, about 14 mM, about 15 mM, about 16 mM, about 17 mM, about 18 mM, about 19 mM, or about 20 mM, or any value in between of acetate and/or a pharmaceutically acceptable acetate salt. In preferred embodiments, the composition comprises about 15 mM of acetate or a pharmaceutically acceptable acetate salt.

In certain embodiments, the composition comprises about 6% (w/v) to about 10% (w/v) or about 7% (w/v) to about 9% (w/v) of sucrose. The composition can, for example comprise about 6% (w/v), about 7% (w/v), about 8% (w/v), about 9% (w/v), or about 10% (w/v), or any value in between of sucrose. In an embodiment, the composition comprises about 7.8% (w/v) of sucrose.

In certain embodiments, the composition comprises about 16 mg/mL to about 24 mg/mL or about 18 mg/mL to about 22 mg/mL of EDTA. The composition can, for example, comprise about 16 mg/mL, about 17 mg/mL, about 18 mg/mL, about 19 mg/mL, about 20 mg/mL, about 21 mg/mL, about 22 mg/mL, about 23 mg/mL, or about 24 mg/mL, or any value in between of EDTA. In particular embodiments, the composition comprises about 20 mg/ml of EDTA.

In certain embodiments, the composition comprises about 0.01% to about 0.07%, about 0.02% to about 0.06%, or about 0.03% to about 0.05% of polysorbate 20 (PS 20) (w/v). The composition can, for example, comprise about 0.01%, about 0.02%, about 0.03%, about 0.04%, about 0.05%, about 0.06%, or about 0.07%, or any value in between of PS 20 (w/v). In particular embodiments, the composition comprises about 0.04% PS 20 (w/v). In particular embodiments, the composition comprises about 0.06% PS 20 (w/v). In alternative embodiments, the composition comprises P188 or polysorbate 80 (PS 80) instead of PS 20 (e.g., about 0.01%, about 0.02%, about 0.03%, about 0.04%, about 0.05%, about 0.06%, or about 0.07%, or any value in between of P188 or PS 80 (w/v)).

In certain embodiments, the pH of the composition is about 4.7 to about 5.7, about 4.8 to about 5.6, about 4.9 to about 5.5. The pH of the composition can, for example, be about 4.7 about 4.8, about 4.9, about 5.0, about 5.1, about 5.2, about 5.3, about 5.4, about 5.5, about 5.6, or about 5.7, or any value in between. In preferred embodiments, the pH of the composition is about 5.2.

High-Concentration Aqueous Pharmaceutical Compositions

In certain embodiments, the bispecific BCMA/CD3 antibody has a high concentration that is particularly suitable for treatment doses, wherein the high concentration enables fewer subcutaneous injections of the composition into a subject to achieve a therapeutic dose, compared to a composition having a low concentration of antibody. For example, the antibody may have a concentration of about 100 mg/mL to about 300 mg/mL, about 125 mg/mL to about 275 mg/mL, about 150 mg/mL to about 250 mg/mL, or about 175 mg/mL to about 225 mg/mL. The bispecific BCMA/CD3 antibody can, for example, have a concentration of about 185 mg/mL, about 186 mg/mL, about 187 mg/mL, about 188 mg/mL, about 189 mg/mL, about 190 mg/mL, about 191 mg/mL, about 192 mg/mL, about 193 mg/mL, about 194 mg/mL, about 195 mg/mL, about 196 mg/mL, about 197 mg/mL, about 198 mg/mL, about 199 mg/mL, about 200 mg/mL, about 201 mg/ML, about 202 mg/mL, about 203 mg/mL, about 204 mg/mL, about 205 mg/mL, about 206 mg/mL, about 207 mg/mL, about 208 mg/mL, about 209 mg/mL, about 210 mg/mL, about 211 mg/mL, about 212 mg/mL, about 213 mg/mL, about 214 mg/mL, or about 215 mg/mL, or any value in between. In particular embodiments, the bispecific BCMA/CD3 antibody has a concentration of about 200 mg/mL.

In certain embodiments, the composition comprises about 100 mM Arginine HCl to about 300 mM Arginine HCl, or about 150 mM Arginine HCl to about 250 mM Arginine HCl, or about 175 mM Arginine HCl to about 225 mM Arginine HCl or about 185 mM Arginine HCl to about 215 mM Arginine HCl, or about 190 mM Arginine HCl to about 210 mM Arginine HCl, or any value in between. In a particular embodiment, the composition comprises about 200 mM Arginine HCl.

In certain embodiments, the composition comprises about 10 mM to about 20 mM, about 12 mM to about 18 mM, or about 14 mM to about 16 mM of acetate and/or a pharmaceutically acceptable acetate salt. The composition can, for example, comprise about 10 mM, about 11 mM, about 12 mM, about 13 mM, about 14 mM, about 15 mM, about 16 mM, about 17 mM, about 18 mM, about 19 mM, or about 20 mM, or any value in between of acetate and/or a pharmaceutically acceptable acetate salt. In preferred embodiments, the composition comprises about 15 mM of acetate or a pharmaceutically acceptable acetate salt.

In certain embodiments, the composition comprises about 1% (w/v) to about 7% (w/v) or about 2% (w/v) to about 6% (w/v) of sucrose. The composition can, for example comprise about 1% (w/v), about 2% (w/v), about 3% (w/v), about 4% (w/v), about 5% (w/v), about 6% (w/v), or about 7% (w/v), or any value in between of sucrose. In a particular embodiment, the composition comprises about 4% (w/v) of sucrose.

In certain embodiments, the composition comprises about 16 mg/mL to about 24 mg/mL or about 18 mg/mL to about 22 mg/mL of EDTA. The composition can, for example, comprise about 16 mg/mL, about 17 mg/mL, about 18 mg/mL, about 19 mg/mL, about 20 mg/mL, about 21 mg/mL, about 22 mg/mL, about 23 mg/mL, or about 24 mg/mL, or any value in between of EDTA. In particular embodiments, the composition comprises about 20 mg/mL of EDTA.

In certain embodiments, the composition comprises about 0.01% to about 0.07% (w/v), about 0.02% to about 0.06%, or about 0.03% to about 0.05% of polysorbate 20 (PS 20). The composition can, for example, comprise about 0.01%, about 0.02%, about 0.03%, about 0.04%, about 0.05%, about 0.06%, or about 0.07%, or any value in between of PS 20 (w/v). In particular embodiments, the composition comprises about 0.04% PS 20 (w/v). In particular embodiments, the composition comprises about 0.06% PS 20 (w/v). In alternative embodiments, the composition comprises P188 or polysorbate 80 (PS 80) instead of PS 20 (e.g., about 0.01%, about 0.02%, about 0.03%, about 0.04%, about 0.05%, about 0.06%, or about 0.07%, or any value in between of P188 or PS 80). The abbreviation “PS20” for Polaxamer 20 is also used interchangeably herein with “PS-20” and “PS 20.”

In certain embodiments, the pH of the composition is about 4.7 to about 5.7, about 4.8 to about 5.6, about 4.9 to about 5.5. The pH of the composition can, for example, be about 4.7 about 4.8, about 4.9, about 5.0, about 5.1, about 5.2, about 5.3, about 5.4, about 5.5, about 5.6, or about 5.7, or any value in between. In preferred embodiments, the pH of the composition is about 5.2.

### Stable Aqueous Pharmaceutical Compositions

In certain embodiments, the stability of the presently disclosed aqueous pharmaceutical compositions, also referred to as drug product (DP), is determined based on specific amount or proportion of the BCMAxCD3 antibody and other constituents of the aqueous pharmaceutical composition as provided herein (such as, but not limited to, acetate and/or pharmaceutically acceptable acetate salts, sucrose, L-Arginine, PS20, and EDTA), as well as the assessment of various factors. These factors include but are not limited to the color of the solution, the pH, the turbidity, percentage of purity, number of subvisible particles, percentage of new peak(s), percentage of main component, percentage of high molecular weight species (HMWS), percentage of low molecular weight species (LMWS), percentage of sum of acidic peaks, percentage of sum of basic peaks, protein concentration, percentage of T-cell activation, viscosity and/or percentage of PS20.

In preferred embodiments, the aqueous pharmaceutical composition is a stable aqueous pharmaceutical composition. A stable aqueous pharmaceutical composition as disclosed herein should not be construed to require all the stability factors listed herein but rather at least one, at least two, or at least three or more of those factors. In some embodiments, the stable disclosed aqueous pharmaceutical composition exhibits the following results for at least one, at least two, at least three or more of the factors listed in detail below herein. In the preferred embodiment, the stable aqueous pharmaceutical composition exhibits the following results for most of the factors listed in detail below herein. In the most preferred embodiment, the stable aqueous pharmaceutical composition exhibits the following results for all the factors listed in detail below herein.

### Color of solution

The Color of an aqueous pharmaceutical composition solution is monitored and can be assessed to verify that the appearance of the solution is consistent with previous batches at release and over the shelf life. The color of the aqueous pharmaceutical composition solution can reflect stability. In one embodiment, the stability of the aqueous pharmaceutical composition is defined when having a color of solution spanning from colorless to about BY2 or less, to about BY4 or less, to about B2 or less, to about B4 or less, to about Y2 or less or to about Y4 or less as described in the European Pharmacopoeia 2.2.2,Degree of Coloration of Liquids European Pharmacopocia (Ph. Eur.) 10th Edition monograph number 20202, July 2019.

In one embodiment, the stability is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a color of solution of colorless to about BY4 or less, to about B4 or less, to about Y4 or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

### pH

Measuring the pH of the aqueous pharmaceutical composition allows confirmation that it is consistent with previous batches at release and over the shelf life. In one embodiment, the stability of the aqueous pharmaceutical composition is defined when its pH is about: 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0. In one embodiment, the pH of the aqueous pharmaceutical composition is about 5.2 after storage for about 12 months or more and at a temperature of about 5° C., after storage for about 12 months or more and at a temperature of about 25° C., and/or after storage for about 2 years or more and at a temperature of about 5° C. In one embodiment, the pH ranges from about 4.7 to about 5.7 after storage for about 12 months or more and at a temperature of about 5° C., after storage for about 12 months or more and at a temperature of about 25° C., and/or after storage for about 2 years or more and at a temperature of about 5° C. In a preferred embodiment, the stability of the aqueous pharmaceutical composition is defined when its pH ranges from about 4.8 to about 5.6 after storage for about 12 months or more and at a temperature of about 5° C., after storage for about 12 months or more and at a temperature of about 25° C., and/or after storage for about 2 years or more and at a temperature of about 5° C. In a most preferred embodiment, the stability of the aqueous pharmaceutical composition is defined when its pH ranges from about 4.9 to about 5.5 after storage for about 12 months or more and at a temperature of about 5° C., after storage for about 12 months or more and at a temperature of about 25° C., and/or after storage for about 2 years or more and at a temperature of about 5° C.

### Turbidity

Turbidity allows measuring the presence of particles in the aqueous pharmaceutical composition in order to ensure consistency with previous batches and applicable compendia guidance at release and over the shelf life. In one embodiment, the stability of the aqueous pharmaceutical composition is defined when its turbidity value is about: 0, 1, 2, 3, 4, 5, 6, 7, 8, 9, 10, 11, 12, 13, 14, 15, 16, 17, 18, 19, or 20 nephelometric turbidity units (NTU) after storage for about 12 months or more and at a temperature of about 5° C., after storage for about 12 months or more and at a temperature of about 25° C., and/or after storage for about 2 years or more and at a temperature of about 5° C. In one embodiment, the stability of the aqueous pharmaceutical composition is defined when its turbidity value is about or less than 18 NTU after storage for about 12 months or more and at a temperature of about 5° C., after storage for about 12 months or more and at a temperature of about 25° C., and/or after storage for about 2 years or more and at a temperature of about 5° C. In a preferred embodiment, the stability of the aqueous pharmaceutical composition is defined when its turbidity value is about or less than 13 NTU after storage for about 12 months or more and at a temperature of about 5° C., after storage for about 12 months or more and at a temperature of about 25° C., and/or after storage for about 2 years or more and at a temperature of about 5° C. In the most preferred embodiment, the stability of the aqueous pharmaceutical composition is defined when its turbidity value is about or less than 8 NTU after storage for about 12 months or more and at a temperature of about 5° C., after storage for about 12 months or more and at a temperature of about 25° C., and/or after storage for about 2 years or more and at a temperature of about 5° C.

### Particle Analysis

The stability of the aqueous pharmaceutical composition is set to a specific threshold of particles contamination based on the average number of sub-visible particles. In one embodiment, the average number of particles present in the units tested should not exceed 100, 200, 300, 400, 500, 600, 700, 800, 900, 1000, 2000, 3000, 4000, 5000, or 6000, per container for particle size equal to 10 μm or greater. In one embodiment, the average number of particles present in the units tested should not exceed 6000 per container for particle size equal to 10 μm or greater. In one embodiment, the average number of particles present in the aqueous pharmaceutical composition units tested should not exceed 100, 200, 300, 400, 500, or 600, per container for particle size equal to 25 μm or greater. In one embodiment, the average number of particles present in the units tested should not exceed 600 per container for particle size equal to 25 μm or greater.

### cSDS Conditions

Capillary SDS-PAGE (cSDS), non-reduced like gel-based SDS-PAGE, is a method for separating denatured protein based on molecular weight. This process allows quantifying DP purity and monitoring its stability at release and over the shelf life.

In one embodiment, the aqueous pharmaceutical composition stability is defined based upon various results of cSDS variables (e.g. percent purity or presence of new peak) where the cSDS was performed under reduced or non-reduced conditions after storage for about 12 months or more and at a temperature of about 5° C., after storage for about 12 months or more and at a temperature of about 25° C., and/or after storage for about 2 years or more and at a temperature of about 5° C.

In one embodiment, the DP stability is defined as having a percent purity about: 80%, 81%, 82%, 83%, 84%, 85%, 86%, 87%, 88%, 89%, 90%, 91%, 92%, 93%, 94%, 95%, 96%, 97%, 98%, 99% or about or equal to 100% or any range in between.

In one embodiment, the DP stability is defined as showing no new peak in the cSDS results of more than 0.5%, 0.8%, 0.9%, 1.0%, 1.2%, 1.3%, 1.4%, 1.5%, 1.6%, 1.7%, 1.8%, 1.9% or more than 2% when compared to an untreated reference material.

In one embodiment, the DP stability is defined with a percent purity of about 90% or more and no new peak of more than 1.5% as compared to a reference material. In a preferred embodiment, the DP stability is defined with a percent purity of about 95% or more, and with no new peak of more than 1.2% compared to a reference material. In a most preferred embodiment, the DP stability is defined as having a percent purity of about 97% or more and with no new peak of more than 1.0% as compared to a reference material.

As used herein, a reference material refers to a material produced in a controlled environment that is used as a calibrator to produce additional substances or materials (e.g., materials of documented purity certified by an analytical laboratory or other noncommercial establishment). In many cases, reference materials are publicly available, such as commercially available drug product (e.g., teclistamab is marketed as TECVAYLI®).

### Size-Exclusion HPLC (SE-HPLC) Results Consistent with Stability

SE-HPLC procedure allows assessing purity of the DP and monitoring its stability under non-denaturing conditions at release and over the shelf life.

In one embodiment, the DP stability is defined based upon various results of SE-HPLC variables such as the Main Component (MC), High Molecular Weight Species (HMWS), or Low Molecular Weight Species (LMWS), after storage of the DP for about 12 months or more and at a temperature of about 5° C., after storage for about 12 months or more and at a temperature of about 25° C., and/or after storage for about 2 years or more and at a temperature of about 5° C. As used herein, “main component” refers to the antibody monomer (i.e., the monomeric species of the anti.

In one embodiment, the DP stability is defined as having a MC of about: 80%, 81%, 82%, 83%, 84%, 85%, 86%, 87%, 88%, 89%, 90%, 91%, 92%, 93%, 94%, 95%, 96%, 97%, 98%, 99% or equal to about 100% or any range in between. In one embodiment, the DP stability is defined as having a MC of about 90% or more. In a preferred embodiment, the DP stability is defined as having a MC of about 95% or more. In the most preferred embodiment, the DP stability is defined as having a MC of about 97% or more.

In one embodiment, the DP stability is defined as having a HMWS of about: 0.1%, 0.5%, 1%, 2%, 3%, 4%, 5%, 6%, 7%, 8%, 9%, 10%, 11%, 12%, 13%, 14%, 15% or any range in between. In one embodiment, the DP stability is defined as having a HMWS of about 10% or less. In a preferred embodiment, the DP stability is defined as having a HMWS of about 5% or less. In the most preferred embodiment, the DP stability is defined as having a HMWS of about to 3% or less.

In one embodiment, the DP stability is defined as having a LMWS of about: 0.1%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%. In one embodiment, the DP stability is defined as having a LMWS of about 5% or less. In a preferred embodiment, the DP stability is defined as having a LMWS of about 2% or less. In a most preferred embodiment, the DP stability is defined as having a LMWS of about 1% or less.

### Capillary Isoelectric Focusing (cIEF)

The cIEF, like isoelectric gel electrophoresis (IEF) methods, separates proteins on the basis of overall charge or isoelectric point (pI). This procedure allows monitoring the distribution of charge-based isoforms of the drug product at release and over the shelf life. In one embodiment, the DP stability is defined based upon various results of cIEF variables such as the Main Peak (MP), the sum of acidic peaks or the sum of basic peaks, after DP storage for about 12 months or more and at a temperature of about 25° C., and/or after storage for about 2 years or more and at a temperature of about 5° C.

In one embodiment, the DP stability is defined as having a cIEF with a MP of about: 60%, 65%, 70%, 75%, 80%, 85%, 90%, 95%, or 100% or any range in between after DP storage for about 12 months or more and at a temperature of about 25° C., and/or after storage for about 2 years or more and at a temperature of about 5° C. In one embodiment, the DP stability is defined as having a cIEF with a MP≥60% after DP storage for about 12 months or more and at a temperature of about 25° C., and/or after storage for about 2 years or more and at a temperature of about 5° C. In a preferred embodiment, the DP stability is defined as having a cIEF with a MP≥65% after DP storage for about 12 months or more and at a temperature of about 25° C., and/or after storage for about 2 years or more and at a temperature of about 5° C. In the most preferred embodiment, the DP stability is defined as having a cIEF with a MP≥70% after DP storage for about 12 months or more and at a temperature of about 25° C., and/or after storage for about 2 years or more and at a temperature of about 5° C.

In one embodiment, the DP stability is defined as having a cIEF with a with a sum of acidic peaks totaling to about: 1%, 5%, 10%, 15%, 20%, 25%, 30%, 35%, 40%, or any range therein between after DP storage for about 12 months or more and at a temperature of about 25° C., and/or after storage for about 2 years or more and at a temperature of about 5° C. In one embodiment, the DP stability is defined as having a cIEF with a sum of acidic peaks ≤40% after DP storage for about 12 months or more and at a temperature of about 25° C., and/or after storage for about 2 years or more and at a temperature of about 5° C. In a preferred embodiment, the DP stability is defined as having a cIEF with a sum of acidic peaks totaling to about ≤ 30% after DP storage for about 12 months or more and at a temperature of about 25° C., and/or after storage for about 2 years or more and at a temperature of about 5° C. In a most preferred embodiment, the DP stability is defined as having a cIEF with a sum of acidic peaks totaling to about ≤ 25% after DP storage for about 12 months or more and at a temperature of about 25° C., and/or after storage for about 2 years or more and at a temperature of about 5° C.

In one embodiment, the DP stability is defined as having a cIEF with a sum of basic peaks totaling about: 1%, 2%, 3%, 4%, 5%, 6%, 7%, 8%, 9%, 10%, 11%, 12%, 13%, 14%, 15%, 16%, 17%, 18%, 19%, or 20%, or any range in between after DP storage for about 12 months or more and at a temperature of about 25° C., and/or after storage for about 2 years or more and at a temperature of about 5° C. In one embodiment, the DP stability is defined as having a cIEF with a sum of basic peaks totaling about 15% or less after DP storage for about 12 months or more and at a temperature of about 25° C., and/or after storage for about 2 years or more and at a temperature of about 5° C. In a preferred embodiment, the DP stability is defined as having a cIEF with a sum of basic peaks totaling less than or about 10% after DP storage for about 12 months or more and at a temperature of about 25° C., and/or after storage for about 2 years or more and at a temperature of about 5° C. In a most preferred embodiment, the DP stability is defined as having a cIEF with a sum of basic peaks totaling less than or about 8% after DP storage for about 12 months or more and at a temperature of about 25° C., and/or after storage for about 2 years or more and at a temperature of about 5° C.

### Protein Concentration

Protein concentration of the DP allows verifying that it is consistent with previous DP batches at release and over the shelf life. Quantification of protein concentration can be accomplished by measuring the UV light absorbance of the drug product solution at 280 nm (A280).

### Stability of Low-Concentration Aqueous Pharmaceutical Compositions

In one embodiment, the DP stability is defined as having a protein concentration of about: 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, or any value in between, after DP storage for about 12 months or more and at a temperature of about 25° C., and/or after storage for about 1 year or more at a temperature of about 5° C. (e.g., 2-8° C.), or about 2 years or more and at a temperature of about 5° C. (e.g., 2-8° C.) (as determined by A280).

In certain embodiments, for protein concentrations of about 5 mg/mL to about 15 mg/mL (e.g., about 10 mg/mL), the total volume of the aqueous pharmaceutical composition (or DP) ranges from about 5 mL to about 10 mL. In one embodiment, the total volume of the aqueous pharmaceutical composition (or DP) ranges from about 0.5 mL to about 20 mL, from about 1 mL to about 15 mL, from about 5 mL to about 10 mL, or from about 6 mL to about 8 mL. In one embodiment, the total volume of the aqueous pharmaceutical composition is about: 0.5 mL, 0.6 mL, 0.7 mL, 0.8 mL, 0.9 mL, 1 mL, 2 mL, 3 mL, 4 mL, 5 mL, 6 mL, 8 mL, 9 mL, 10 mL, 11 mL, 12 mL, 13 mL, 14 mL, 15 mL, 16 mL, 18 mL, 19 mL, 20 mL, 25 mL, or 30 mL or any range in between. In one embodiment, the total volume of the aqueous pharmaceutical composition is about 2 mL; for example, about 10 mg antibody/mL in a 3.0 mL vial (about 30 mg antibody/vial).

In one embodiment, the DP stability is defined as having a protein concentration of about 7 mg/mL to about 13 mg/mL after DP storage for about 12 months or more and at a temperature of about 25° C., and/or after storage for about 1 year or more at a temperature of about 5° C. (e.g., 2-8° C.), or about 2 years or more and at a temperature of about 5° C. (e.g., 2-8° C.) (as determined by A280). In a preferred embodiment, the DP stability is defined as having a protein concentration of about 8 mg/mL to about 12 mg/mL after DP storage for about 12 months or more and at a temperature of about 25° C., and/or after storage for about 1 year or more at a temperature of about 5° C. (e.g., 2-8° C.), or about 2 years or more and at a temperature of about 5° C. (e.g., 2-8° C.) (as determined by A280). In a most preferred embodiment, the DP stability is defined as having a protein concentration of about 9 mg/mL to 11 mg/mL after DP storage for about 12 months or more and at a temperature of about 25° C., and/or after storage for about 1 year or more at a temperature of about 5° C. (e.g., 2-8° C.), or about 2 years or more and at a temperature of about 5° C. (e.g., 2-8° C.) (as determined by A280).

### Stability of High-Concentration Aqueous Pharmaceutical Compositions

In one embodiment, the DP stability is defined as having a protein concentration of about 185 mg/mL, about 186 mg/mL, about 187 mg/mL, about 188 mg/mL, about 189 mg/mL, about 190 mg/mL, about 191 mg/mL, about 192 mg/mL, about 193 mg/mL, about 194 mg/mL, about 195 mg/mL, about 196 mg/mL, about 197 mg/mL, about 198 mg/mL, about 199 mg/mL, about 200 mg/mL, about 201 mg/ML, about 202 mg/mL, about 203 mg/mL, about 204 mg/mL, about 205 mg/mL, about 206 mg/mL, about 207 mg/mL, about 208 mg/mL, about 209 mg/mL, about 210 mg/mL, about 211 mg/mL, about 212 mg/mL, about 213 mg/mL, about 214 mg/mL, or about 215 mg/mL, or any value in between, after DP storage for about 12 months or more and at a temperature of about 25° C., and/or after storage for about 1 year or more at a temperature of about 5° C. (e.g., 2-8° C.), or about 2 years or more and at a temperature of about 5° C. (e.g., 2-8° C.) (as determined by A280).

In certain embodiments, for protein concentrations of about 185 mg/mL to about 215 mg/mL (e.g., about 200 mg/mL), the total volume of the aqueous pharmaceutical composition (or DP) ranges from about 0.5 mL to about 2 mL. In one embodiment, the total volume of the aqueous pharmaceutical composition (or DP) ranges from about 0.5 mL to about 2 mL, from about 1 mL to about 2 mL, from about 1.25 mL to about 1.75 mL, or from about 1.4 mL to about 1.6 mL. In one embodiment, the total volume of the aqueous pharmaceutical composition is about: 0.5 mL, 0.6 mL, 0.7 mL, 0.8 mL, 0.9 mL, 1 mL, 1.1 mL, 1.2 mL, 1.3 mL, 1.4 mL, 1.5 mL, 1.6 mL, 1.7 mL, 1.8 mL, 1.9 mL, or 2 mL or any range in between. In one embodiment, the total volume of the aqueous pharmaceutical composition is about 1.5 mL; for example, about 200 mg antibody/mL in a 1.5 mL vial (about 300 mg antibody/vial).

In one embodiment, the DP stability is defined as having a protein concentration of about 175 mg/mL to about 225 mg/mL after DP storage for about 12 months or more and at a temperature of about 25° C., and/or after storage for about 1 year or more at a temperature of about 5° C. (e.g., 2-8° C.), or about 2 years or more and at a temperature of about 5° C. (e.g., 2-8° C.) (as determined by A280). In a preferred embodiment, the DP stability is defined as having a protein concentration of about 180 mg/mL to about 220 mg/mL after DP storage for about 12 months or more and at a temperature of about 25° C., and/or after storage for about 1 year or more at a temperature of about 5° C. (e.g., 2-8° C.), or about 2 years or more and at a temperature of about 5° C. (e.g., 2-8° C.) (as determined by A280). In another preferred embodiment, the DP stability is defined as having a protein concentration of about 190 mg/mL to 210 mg/mL after DP storage for about 12 months or more and at a temperature of about 25° C., and/or after storage for about 1 year or more at a temperature of about 5° C. (e.g., 2-8° C.), or about 2 years or more and at a temperature of about 5° C. (e.g., 2-8° C.) (as determined by A280).

According to an embodiment, the aqueous pharmaceutical composition has a protein concentration of the BCMAxCD3 bispecific antibody of about 180 mg/mL to about 220 mg/mL (preferably about 200 mg/mL) for a shelf life of about 6 months at 5° C. (e.g., 2-8° C.), or for about 12 months at 5° C. (e.g., 2-8° C.), or for about 18 months at 5° C. (e.g., 2-8° C.), or for about 24 months at 5° C. (e.g., 2-8° C.), or for about 30 months at 5° C. (e.g., 2-8° C.), or for about 36 months at 5° C. (e.g., 2-8° C.). According to an embodiment, the aqueous pharmaceutical composition has a protein concentration of the BCMAxCD3 bispecific antibody of about 180 mg/mL to about 220 mg/mL (preferably about 200 mg/mL) for a shelf life of at least about 6 months at 5° C. (e.g., 2-8° C.), or for at least about 12 months at 5° C. (e.g., 2-8° C.), or for at least about 18 months at 5° C. (e.g., 2-8° C.), or for at least about 24 months at 5° C. (e.g., 2-8° C.), or for at least about 30 months at 5° C. (e.g., 2-8° C.), or for at least about 36 months at 5° C. (e.g., 2-8° C.).

According to an embodiment, the aqueous pharmaceutical composition comprises about 180 mg/mL to about 220 mg/mL (e.g., about 200 mg/mL) of the BCMAxCD3 bispecific antibody in 15 mM acetate, 4.0% (w/v) sucrose, 200 mM Arginine-HCl, 20 μg/mL EDTA, 0.04% (w/v) polysorbate 20, at pH 5.2. The composition is preferably contained in a vial, wherein the nominal fill volume of the vial is about 1.5 mL, so the nominal antibody amount is about 300 mg/vial. The preferred temperature condition is 5±3° C. (2-8° C.), and the vial is preferably protected from light. The composition is preferably low-viscosity (e.g., no more than about 25 centipoise (cP), or no more than about 22 cP. or no more than about 20 cP, or no more than about 18 cP, or no more than about 17 cP, most preferably about 16.6 cP), wherein the composition is suitable for subcutaneous administration to a human subject for the treatment of a hematological cancer, such as multiple myeloma.

### Peptide Mapping

Post-translational modifications (PTMs), such as oxidation, deamidation, and isomerization, are enzymatic modifications that may be detected in the structure of an antibody. In some embodiments, the PD stability is assessed based on level of PTMs in the antibody. Test articles are enzymatically digested to yield peptide segments. These peptides are then evaluated by for instance by mass spectrometry (MS), by tandem mass spectrometry (MS-MS) or Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

### Drug Product potency

In vitro T-cell activation assay allows assessing the level of DP stability. This activation can be assessed by using, but not limited to, a Nuclear factor of activated T cells-Response Element (NFAT-RE)-mediated luminescence assay.

### BCMAxCD3 T-cell Activation Activity

In one embodiment, the DP stability is defined as having a BMCAxCD3-mediated T-cell activation activity, relative to a reference, of about: 40%, 50%, 60%, 70%, 80%, 90%, 100%, 110%, 120%, 130%, 140%, 150%, or 160% or any range in between after DP storage for about 12 months or more and at a temperature of about 5° C. (e.g., 2-8° C.), after storage for about 12 months or more and at a temperature of about 25° C., and/or after storage for about 1 year or more or about 2 years or more at a temperature of about 5° C. (e.g., 2-8° C.). In one embodiment, the DP stability is defined as having an BMCAxCD3-mediated T-cell activation activity ranging from about 50% to about 150% relative to a reference after DP storage for about 12 months or more and at a temperature of about 25° C., and/or after storage for about 1 year or more or about 2 years or more at a temperature of about 5° C. (e.g., 2-8° C.). In a preferred embodiment, the DP stability is defined as having an BMCAxCD3-mediated T-cell activation activity ranging from about 60% to about 140% relative to a reference after DP storage for about 12 months or more and at a temperature of about 25° C., and/or after storage for about 1 year or more or about 2 years or more at a temperature of about 5° C. (e.g., 2-8° C.). In a most preferred embodiment, the DP stability is defined as having an BMCAxCD3-mediated T-cell activation activity ranging from about 80% to about 120% relative to a reference after DP storage for about 12 months or more and at a temperature of about 25° C., and/or after storage for about 1 year or more or about 2 years or more and at a temperature of about 5° C. (e.g., 2-8° C.).

### Polysorbate 20 (PS20)

In one embodiment, the DP stability is defined by a PS20 concentration in percentage weight to volume of about: 0.005%, 0.01%, 0.02%, 0.03%, 0.04%, 0.05%, 0.06%, 0.07%, 0.08%, 0.09%, 0.10%, or any range in between after DP storage for about 12 months or more and at a temperature of about 5° C., after storage for about 12 months or more and at a temperature of about 25° C., and/or after storage for about 2 years or more and at a temperature of about 5° C. In one embodiment, the DP stability is defined by a PS20 concentration of about 0.02% to about 0.1% after DP storage for about 12 months or more and at a temperature of about 25° C., and/or after storage for about 2 years or more and at a temperature of about 5° C. In one embodiment, the DP stability is defined by a PS20 concentration of about 0.01% to about 0.07%. In a preferred embodiment, the DP stability is defined by a PS20 concentration of about 0.02% to about 0.06% after DP storage for about 12 months or more and at a temperature of about 25° C., and/or after storage for about 2 years or more and at a temperature of about 5° C. In a most preferred embodiment, the DP stability is defined with a PS20 concentration of about 0.03% to about 0.05% after DP storage for about 12 months or more and at a temperature of about 25° C., and/or after storage for about 2 years or more and at a temperature of about 5° C.

### Embodiments of High-Concentration, Low-Viscosity Pharmaceutical Compositions

The inventors have discovered that a BCMA/CD3 bispecific antibody as described herein can be formulated into a low-viscosity, high-concentration (e.g., about 150 mg/mL to about 250 mg/mL, or about 180 mg/mL to about 220 mg/mL, or about 200 mg/mL antibody concentration) composition that is suitable for subcutaneous administration to a human subject for the treatment of a hematological cancer, such as multiple myeloma.

According to an embodiment, the composition comprises a viscosity-reducing agent, such as arginine-HCl.

According to a preferred embodiment, the aqueous pharmaceutical composition has a viscosity of about 25 centipoise (cP) or less, more preferably about 24 cP or less, or about 23 cP or less, or about 22 cP or less, or about 22 cP or less, or about 20 cP or less, or about 19 cP or less, or about 18 cP or less, or about 17 cP or less at 25° C. According to an embodiment, the pharmaceutical composition has a viscosity from about 12 cP to about 22 cP, or from about 13 cP to about 21 cP, or from about 14 cP to about 20 cP, or from about 15 cP to about 19 cP, or from about 12 cP to about 20 cP, or from about 15 cP to about 18 cP, or from about 15 cP to about 17 cP at 25° C. According to certain embodiments, the composition has a viscosity of about 16.6 cP at 25° C.

According to certain embodiments, an aqueous pharmaceutical composition comprising a BCMA/CD3 bispecific antibody as described herein can be formulated to have a high-concentration (e.g., about 150 mg/mL to about 250 mg/mL, or about 180 mg/mL to about 220 mg/mL, or about 200 mg/mL antibody concentration) with low-viscosity (e.g., no more than about 25 centipoise (cP), or no more than about 22 cP. or no more than about 20 cP, or no more than about 18 cP, or no more than about 17 cP), wherein the composition is suitable for subcutaneous administration to a human subject for the treatment of a hematological cancer, such as multiple myeloma.

According to an embodiment, an aqueous pharmaceutical composition comprises: (a) a concentration of about 150 mg/mL to about 250 mg/mL (e.g., about 180 mg/mL to about 220 mg/mL, or about 200 mg/mL) of a bispecific BCMA/CD3 antibody as described herein; and (b) a viscosity reducing agent (preferably, arginine HCl), wherein the composition has a pH of about 4.7 to about 5.7, and is suitable for subcutaneous administration to a human subject for the treatment of a hematological cancer, such as multiple myeloma, and wherein the composition has one or more of the following features:
(a) a color of solution that is colorless to ≤BY2, ≤B2, ≤Y2;
(b) an osmolality of about 612 to about 828 mOsm/kg;
(c) BMCAxCD3-mediated T-cell activation activity ranging from about 60% to about 140% relative to teclistamab reference material;
(d) a density of about 1.079 g/mL at 20-25° C.;
(e) a viscosity of about 15 to about 18 cP (e.g., about 16.6 cP) at 25° C.;
(f) a purity of ≥95.0%, and no new peak >1.0% compared to teclistamab reference material, as determined by cSDS (reduced);
(g) a purity of ≥90.0%, and no new peak >1.0% compared to teclistamab reference material, as determined by cSDS (non-reduced); and/or
(h) a main component of ≥95.0%, HMWS of ≤5.0%, and LMWS of <5.0%, as determined by SE-HPLC (or a main component of ≥90.0%, HMWS of ≤10.0%, and LMWS of <10.0%, as determined by SE-HPLC).

According to a preferred embodiment, the aqueous pharmaceutical composition has a viscosity of about 25 centipoise (cP) or less, more preferably about 24 cP or less, or about 23 cP or less, or about 22 cP or less, or about 22 cP or less, or about 20 cP or less, or about 19 cP or less, or about 18 cP or less, or about 17 cP or less. According to an embodiment, the pharmaceutical composition has a viscosity from about 12 cP to about 22 cP, or from about 13 cP to about 21 cP, or from about 14 cP to about 20 cP, or from about 15 cP to about 19 cP, or from about 12 cP to about 20 cP, or from about 15 cP to about 18 cP, or from about 15 cP to about 17 cP. According to certain embodiments, the composition has a viscosity of about 16.6 cP at 25° C.

### Methods of the Present Invention

Provided herein are methods of treating cancer in a subject in need thereof comprising administering to the subject an aqueous pharmaceutical composition as disclosed herein. In certain embodiments, the administration is subcutaneous.

### Cancers

In some embodiments, the cancer is a hematological malignancy or a solid tumor.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In particular embodiments, the hematological malignancy is multiple myeloma. In some embodiments, the subject has a newly diagnosed multiple myeloma. In some embodiments, the subject is relapsed or refractory to treatment with one or more prior anti-cancer treatments, such as a therapeutic used to treat multiple myeloma or other hematological malignancies.

In some embodiments, the subject is refractory or relapsed to treatment with one or more treatments or therapies, such as THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (42elinexor42at), AREDIA® (pamidronate), ZOMETA® (zoledronic acid), DARZALEX® (daratumumab), elotozumab or melphalan, Xpovio® (Selinexor), Venclexta® (Venetoclax), GSK 916, CAR-T therapies, other BCMA-directed therapies.

Various qualitative and/or quantitative methods can be used to determine relapse or refractory nature of the disease. Symptoms that can be associated are for example a decline or plateau of the well-being of the patient or re-establishment or worsening of various symptoms associated with solid tumors, and/or the spread of cancerous cells in the body from one location to other organs, tissues or cells.

In some embodiments, the multiple myeloma is relapsed or refractory to treatment with an anti-CD38 antibody, selinexor, venetoclax, lenalinomide, bortezomib, pomalidomide, carfilzomib, elotozumab, ixazomib, melphalan or thalidomide, or any combination thereof.

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4;14)(p16;q32), t(14;16)(q32;q23), del17p, 1 qAmp, t(4;14)(p16;q32) and t(14;16)(q32;q23), t(4;14)(p16;q32) and del17p, t(14;16)(q32;q23) and del17p, or t(4;14)(p16;q32), t(14; 16)(q32;q23) and del17p. In some embodiments, the subject having the high-risk multiple myeloma has one or more chromosomal abnormalities comprising: t(4;14)(p16;q32), t(14;16)(q32;q23), del17p, 1qAmp, t(4;14)(p16;q32) and t(14;16)(q32;q23), t(4; 14)(p16;q32) and del17p, t(14;16)(q32;q23) and del17p; or t(4;14)(p16;q32), t(14;16)(q32;q23) and del17p, or any combination thereof.

The cytogenetic abnormalities can be detected for example by fluorescent in situ hybridization (FISH). In chromosomal translocations, an oncogene is translocated to the IgH region on chromosome 14q32, resulting in dysregulation of these genes. T(4;14)(p16;q32) involves translocation of fibroblast growth factor receptor 3 (FGFR3) and multiple myeloma SET domain containing protein (MMSET)(also called WHSC1/NSD2), and t(14;16)(q32;q23) involves translocation of the MAF transcription factor C-MAF. Deletion of 17p (del17p) involves loss of the p53 gene locus.

Chromosomal rearrangements can be identified using well known methods, for example fluorescent in situ hybridization, karyotyping, pulsed field gel electrophoresis, or sequencing.

In an embodiment, a method of treating a hematological cancer, such as multiple myeloma, in a subject in need thereof, comprises subcutaneously administering to the subject one or more step-up doses of a composition A, wherein composition A comprises about 10 mg/mL of a bispecific BCMA/CD3 antibody, about 15 mM of acetate and/or pharmaceutically acceptable acetate salt, about 7.8% (w/v) sucrose, about 10 mM Arginine HCL, about 20 μg/mL EDTA, and about 0.04% PS 20, wherein composition A has a pH of about 5.2;
and then subcutaneously administering to the subject a composition B once per week, wherein composition B comprises about 200 mg/mL of the bispecific BMCA/CD3 antibody, about 15 mM of acetate and/or pharmaceutically acceptable acetate salt, about 4% (w/v) sucrose, about 200 mM Arginine HCL, about 20 μg/mL EDTA, and about 0.04% PS 20, wherein composition B has a pH of about 5.2. Preferably, the bispecific BMCA/CD3 antibody comprises a HC2 having at least 95% sequence identity to the amino acid sequence of SEQ ID NO:19, and a LC2 having at least 95% sequence identity to the amino acid sequence of SEQ ID NO:20.

Also provided herein is a method for preparing an aqueous pharmaceutical composition of a bispecific B-cell mature antigen (BCMA)/cluster of differentiation 3 (CD3) antibody or antigen-binding fragment thereof, the bispecific BCMA/CD3 antibody or antigen-binding fragment thereof comprising: a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID Nos:1, 2, and 3, respectively; a first light chain (LC1) comprising a LC1 variable region (VL1), wherein the VL1 comprises a light chain complementarity determining region 1 (LCDR1), a LCDR2, and a LCDR3 having the amino acid sequences of SEQ ID Nos:4, 5, and 6, respectively; a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID Nos:11, 12, and 13, respectively; and a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID Nos:14, 15, and 16, respectively; the method comprising combining the bispecific BCMA/CD3 antibody with acetate and/or a pharmaceutically acceptable acetate salt, arginine HCl, sucrose, EDTA, and polysorbate (PS) 20 in amounts described herein, wherein the aqueous pharmaceutical composition has a pH of about 5.2.

In certain embodiments, the bispecific BCMA/CD3 antibody comprises a VH1 having the amino acid sequence of SEQ ID NO:7 and a VL1 having the amino acid sequence of SEQ ID NO:8. In certain embodiments, the bispecific BCMA/CD3 antibody comprises a HC1 having the amino acid sequence of SEQ ID NO:9, and a LC1 having the amino acid sequence of SEQ ID NO:10. In certain embodiments, the bispecific BCMA/CD3 antibody comprises a VH2 having the amino acid sequence of SEQ ID NO:17, and a VL2 having the amino acid sequence of SEQ ID NO:18. In certain embodiments, the bispecific BCMA/CD3 antibody comprises a HC2 having the amino acid sequence of SEQ ID NO:19, and a LC2 having the amino acid sequence of SEQ ID NO:20. The bispecific BCMA/CD3 antibody can, for example, be teclistamab.

The aqueous pharmaceutical compositions disclosed herein can be packaged into kits, containers, packs, dispensers, or vials.

Provided herein is a kit comprising the disclosed aqueous pharmaceutical and instructions for use thereof.

Also provided herein is an article of manufacture comprising a container holding the disclosed aqueous pharmaceutical composition. In some embodiments, the container is a vial with a stopper pierceable by a syringe.

### EMBODIMENTS

Provided here are illustrative embodiments of the disclosed technology. These embodiments are illustrative only and do not limit the scope of the present disclosure or of the claims attached hereto.

1. An aqueous pharmaceutical composition comprising:
a) about 150 mg/mL to about 250 mg/mL of a bispecific B-cell mature antigen (BCMA)/cluster of differentiation 3 (CD3) antibody, the bispecific BCMA/CD3 antibody comprising:
   (5) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (6) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (7) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (8) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively; and
b) arginine HCl,
wherein the composition has a viscosity of no more than about 25 centipoise (cP) at 25° C.
   2. The aqueous pharmaceutical composition of embodiment 1, wherein the bispecific BCMA/CD3 antibody comprises a VH1 having the amino acid sequence of SEQ ID NO:7, and a VL1 having the amino acid sequence of SEQ ID NO:8.
   3. The aqueous pharmaceutical composition of embodiment 1 or embodiment 2, wherein the bispecific BCMA/CD3 antibody comprises a VH2 having the amino acid sequence of SEQ ID NO:17, and a VL2 having the amino acid sequence of SEQ ID NO:18.
   4. The aqueous pharmaceutical composition of any of embodiments 1-3, wherein the bispecific BCMA/CD3 antibody comprises a HC1 having at least 95% sequence identity to the amino acid sequence of SEQ ID NO:9, and a LC1 having at least 95% sequence identity to the amino acid sequence of SEQ ID NO:10.
   5. The aqueous pharmaceutical composition of any of embodiments 1-3, wherein the bispecific BCMA/CD3 antibody comprises a HC1 having the amino acid sequence of SEQ ID NO:9, and a LC1 having the amino acid sequence of SEQ ID NO:10.
   6. The aqueous pharmaceutical composition of any of embodiments 1-5, wherein the bispecific BCMA/CD3 antibody comprises a HC2 having at least 95% sequence identity to the amino acid sequence of SEQ ID NO:19, and a LC2 having at least 95% sequence identity to the amino acid sequence of SEQ ID NO:20.
   7. The aqueous pharmaceutical composition of any of embodiments 1-5, wherein the bispecific BCMA/CD3 antibody comprises a HC2 having the amino acid sequence of SEQ ID NO:19, and a LC2 having the amino acid sequence of SEQ ID NO:20.
   8. The aqueous pharmaceutical composition of embodiment 1, wherein the bispecific BCMA/CD3 antibody is teclistamab.
   9. The aqueous pharmaceutical composition of any of embodiments 1-8, wherein the composition comprises about 180 mg/mL to about 220 mg/mL of the bispecific BCMA/CD3 antibody.
   10. The aqueous pharmaceutical composition of any of embodiments 1-8, wherein the composition comprises about 200 mg/mL of the bispecific BCMA/CD3 antibody.
   11. The aqueous pharmaceutical composition of any of embodiments 1-10, wherein the composition comprises about 100 mM to about 300 mM arginine HCl.
   12. The aqueous pharmaceutical composition of any of embodiments 1-10, wherein the composition comprises about 150 mM to about 250 mM arginine HCl.
   13. The aqueous pharmaceutical composition of any of embodiments 1-10, wherein the composition comprises about 200 mM arginine HCl.
   14. The aqueous pharmaceutical composition of any of embodiments 1-13, wherein the composition has a viscosity of no more than about 22 centipoise (cP) at 25° C.
   15. The aqueous pharmaceutical composition of any of embodiments 1-13, wherein the composition has a viscosity of no more than about 20 centipoise (cP) at 25° C.
   16. The aqueous pharmaceutical composition of any of embodiments 1-13, wherein the composition has a viscosity of no more than about 18 centipoise (cP) at 25° C.
   17. The aqueous pharmaceutical composition of any of embodiments 1-13, wherein the composition has a viscosity from about 10 centipoise (cP) to about 20 cP at 25° C.
   18. The aqueous pharmaceutical composition of any of embodiments 1-13, wherein the composition has a viscosity from about 15 centipoise (cP) to about 18 cP at 25° C.
   19. The aqueous pharmaceutical composition of any of embodiments 1-13, wherein the composition has a viscosity from about 16 centipoise (cP) to about 17 cP (e.g., about 16.6 cP) at 25° C.
   20. The aqueous pharmaceutical composition of any of embodiments 1-19, wherein the composition comprises high molecular weight species of the bispecific BCMA/CD3 antibody at no more than about 10% of the bispecific BCMA/CD3 antibody.
   21. The aqueous pharmaceutical composition of any of embodiments 1-19, wherein the composition comprises high molecular weight species of the bispecific BCMA/CD3 antibody at no more than about 7.5% of the bispecific BCMA/CD3 antibody.
   22. The aqueous pharmaceutical composition of any of embodiments 1-19, wherein the composition comprises high molecular weight species of the bispecific BCMA/CD3 antibody at no more than about 5% of the bispecific BCMA/CD3 antibody.
   23. The aqueous pharmaceutical composition of any of embodiments 1-19, wherein the composition has one or more of the following features:
(a) a color of solution that is colorless to ≤BY2, ≤B2, ≤Y2;
   (b) an osmolality of about 612 to about 828 mOsm/kg;
   (c) BMCAxCD3-mediated T-cell activation activity ranging from about 60% to about 140% relative to teclistamab reference material; and/or
   (d) a main component of ≥95.0%, HMWS of ≤5.0%, and LMWS of <5.0%, as determined by SE-HPLC (or a main component of ≥90.0%, HMWS of ≤10.0%, and LMWS of <10.0%, as determined by SE-HPLC).
      24. The aqueous pharmaceutical composition of any of embodiments 1-19, wherein the composition has one or more of the following features:
(a) a color of solution that is colorless to ≤BY2, ≤B2, ≤Y2;
   (b) an osmolality of about 612 to about 828 mOsm/kg;
   (c) BMCAxCD3-mediated T-cell activation activity ranging from about 60% to about 140% relative to teclistamab reference material;
   (d) a purity of ≥95.0%, and no new peak >1.0% compared to teclistamab reference material, as determined by cSDS (reduced);
   (e) a purity of ≥90.0%, and no new peak >1.0% compared to teclistamab reference material, as determined by cSDS (non-reduced); and/or
   (f) a main component of ≥95.0%, HMWS of ≤5.0%, and LMWS of <5.0%, as determined by SE-HPLC (or a main component of ≥90.0%, HMWS of ≤10.0%, and LMWS of <10.0%, as determined by SE-HPLC).
      25. The aqueous pharmaceutical composition of any of embodiments 1-24, wherein the composition is stable.
      26. The aqueous pharmaceutical composition of embodiment 25, wherein stability of the stable composition is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.
      27. The aqueous pharmaceutical composition of embodiment 25 or embodiment 26, wherein the aqueous pharmaceutical composition is stable at a temperature of about 2-8° C. for at least two years.
      28. The aqueous pharmaceutical composition of any of embodiments 1-27, wherein the composition comprises about 180 mg/mL to about 220 mg/mL (preferably about 200 mg/mL) of the BCMAxCD3 bispecific antibody for a shelf life of at least about 6 months at 2-8° C., or for at least about 12 months at 2-8° C., or for at least about 18 months at 2-8° C., or for at least about 24 months at 2-8° C., or for at least about 30 months at 2-8° C., or for at least about 36 months at 2-8° C.
      29. The aqueous pharmaceutical composition of any of embodiments 1-28, wherein the composition is suitable for subcutaneous administration to a human subject for the treatment of a hematological cancer, such as multiple myeloma.
      30. The aqueous pharmaceutical composition of any of embodiments 1-29, wherein the composition comprises about 1% (w/v) to about 7% (w/v) of sucrose, or about 2% (w/v) to about 6% (w/v) of sucrose.
      31. The aqueous pharmaceutical composition of any of embodiments 1-29, wherein the composition comprises about 3% (w/v) to about 5% (w/v) of sucrose.
      32. The aqueous pharmaceutical composition of any of embodiments 1-29, wherein the composition comprises about 4% (w/v) of sucrose.
      33. The aqueous pharmaceutical composition of any of embodiments 1-32, wherein the composition comprises about 10 mM to about 20 mM, or about 12 mM to about 18 mM of acetate and/or pharmaceutically acceptable acetate salt.
      34. The aqueous pharmaceutical composition of any of embodiments 1-32, wherein the composition comprises about 14 mM to about 16 mM of acetate and/or pharmaceutically acceptable acetate salt.
      35. The aqueous pharmaceutical composition of any of embodiments 1-32, wherein the composition comprises about 15 mM of acetate and/or pharmaceutically acceptable acetate salt.
      36. The aqueous pharmaceutical composition of any of embodiments 1-35, wherein the composition comprises about 16 μg/mL to about 24 μg/mL, or about 18 μg/mL to about 22 μg/mL of EDTA.
      37. The aqueous pharmaceutical composition of any of embodiments 1-35, wherein the composition comprises about 20 μg/mL of EDTA.
      38. The aqueous pharmaceutical composition of any of embodiments 1-37, wherein the composition comprises about 0.01% to about 0.07%, or about 0.02% to about 0.06% of PS-20 (w/v).
      39. The aqueous pharmaceutical composition of any of embodiments 1-37, wherein the composition comprises about 0.03 to about 0.05% of PS-20 (w/v).
      40. The aqueous pharmaceutical composition of any of embodiments 1-37, wherein the composition comprises about 0.04% of PS-20 or about 0.06% (w/v).
      41. The aqueous pharmaceutical composition of any of embodiments 1-40, wherein the pH of the composition is about 4.7 to about 5.7, or about 4.8 to about 5.6.
      42. The aqueous pharmaceutical composition of any of embodiments 1-40, wherein the pH of the composition is about 4.9 to about 5.5.
      43. The aqueous pharmaceutical composition of any of embodiments 1-40, wherein the pH of the composition is about 5.2.
      44. The aqueous pharmaceutical composition of any of embodiments 1-40, wherein the composition comprises about 200 mg/mL of the bispecific BCMA/CD3 antibody, about 15 mM of acetate and/or pharmaceutically acceptable acetate salt, about 4% (w/v) sucrose, about 200 mM Arginine HCL, about 20 μg/mL EDTA, and about 0.04% (w/v) PS 20, and the composition has a pH of about 5.2.
      45. A vial containing about 1.5 mL of the composition of embodiment 44, wherein the vial contains about 300 mg of the bispecific BCMA/CD3 antibody.
      46. A vial containing the composition of any of embodiments 1-44.
      47. The vial of embodiment 45 or embodiment 46, wherein the vial comprises a stopper pierceable by a syringe.
      48. A method of treating a hematological cancer, such as multiple myeloma, in a subject in need thereof, comprising subcutaneously administering a therapeutically effective amount of the composition of any of embodiments 1-44 to the subject.
      49. A method of treating a hematological cancer, such as multiple myeloma, in a subject in need thereof, comprising subcutaneously administering a therapeutically effective amount of the composition of any of embodiments 1-44 to the subject as a treatment dose once per week.
      50. A method of treating a hematological cancer, such as multiple myeloma, in a subject in need thereof, comprising subcutaneously administering a therapeutically effective amount of the composition of any of embodiments 1-44 to the subject as a treatment dose once per week, wherein the subject has relapsed/refractory multiple myeloma.
      51. A method of treating a hematological cancer, such as multiple myeloma, in a subject in need thereof, comprising subcutaneously administering to the subject the composition of any of embodiments 1-44 once per week after subcutaneously administering to the subject one or more step-up doses of the same bispecific BCMA/CD3 antibody contained in the composition.
      52. The aqueous pharmaceutical composition of any of embodiments 1-44 for use in the treatment of cancer.
      53. The aqueous pharmaceutical composition of any of embodiments 1-44 for use in the preparation of a medicament for the treatment of cancer.
      54. Use of the aqueous pharmaceutical composition of any of embodiments 1-44 for treating in a subject in need thereof, the use comprising administering the aqueous pharmaceutical composition to the subject in need thereof.
      55. The use of embodiment 54, wherein the administration is subcutaneous.
      56. An aqueous pharmaceutical composition comprising:
a) about 150 mg/mL to about 250 mg/mL of a bispecific B-cell mature antigen (BCMA)/cluster of differentiation 3 (CD3) antibody, the bispecific BCMA/CD3 antibody comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(g) about 10 mM to about 20 mM of acetate and/or pharmaceutically acceptable acetate salt;
(h) about 1% (w/v) to about 7% (w/v) of sucrose;
(i) about 100 mM to about 300 mM arginine HCl;
(j) about 16 μg/mL to about 24 μg/mL of ethylenediaminetetraacetic acid (EDTA); and
(k) about 0.01% to about 0.07% polysorbate 20 (w/v), wherein the composition has a pH from about 4.7 to about 5.7.
   57. The aqueous pharmaceutical composition of embodiment 56, wherein the bispecific BCMA/CD3 antibody comprises a VH1 having the amino acid sequence of SEQ ID NO:7, and a VL1 having the amino acid sequence of SEQ ID NO:8.
   58. The aqueous pharmaceutical composition of embodiment 56 or embodiment 57, wherein the bispecific BCMA/CD3 antibody comprises a VH2 having the amino acid sequence of SEQ ID NO:17, and a VL2 having the amino acid sequence of SEQ ID NO:18.
   59. The aqueous pharmaceutical composition of any of embodiments 56-58, wherein the bispecific BCMA/CD3 antibody comprises a HC1 having at least 95% sequence identity to the amino acid sequence of SEQ ID NO:9, and a LC1 having at least 95% sequence identity to the amino acid sequence of SEQ ID NO:10.
   60. The aqueous pharmaceutical composition of any of embodiments 56-58, wherein the bispecific BCMA/CD3 antibody comprises a HC1 having the amino acid sequence of SEQ ID NO:9, and a LC1 having the amino acid sequence of SEQ ID NO:10.
   61. The aqueous pharmaceutical composition of any of embodiments 56-60, wherein the bispecific BCMA/CD3 antibody comprises a HC2 having at least 95% sequence identity to the amino acid sequence of SEQ ID NO:19, and a LC2 having at least 95% sequence identity to the amino acid sequence of SEQ ID NO:20.
   62. The aqueous pharmaceutical composition of any of embodiments 56-60, wherein the bispecific BCMA/CD3 antibody comprises a HC2 having the amino acid sequence of SEQ ID NO:19, and a LC2 having the amino acid sequence of SEQ ID NO:20.
   63. The aqueous pharmaceutical composition of embodiment 56, wherein the bispecific BCMA/CD3 antibody is teclistamab.
   64. The aqueous pharmaceutical composition of any of embodiments 56-63, wherein the composition comprises about 180 mg/mL to about 220 mg/mL of the bispecific BCMA/CD3 antibody.
   65. The aqueous pharmaceutical composition of any of embodiments 56-63, wherein the composition comprises about 200 mg/mL of the bispecific BCMA/CD3 antibody.
   66. The aqueous pharmaceutical composition of any of embodiments 56-65, wherein the composition comprises about 100 mM to about 300 mM arginine HCl.
   67. The aqueous pharmaceutical composition of any of embodiments 56-65, wherein the composition comprises about 150 mM to about 250 mM arginine HCl.
   68. The aqueous pharmaceutical composition of any of embodiments 56-65, wherein the composition comprises about 200 mM arginine HCl.
   69. The aqueous pharmaceutical composition of any of embodiments 56-68, wherein the composition comprises about 2% (w/v) to about 6% (w/v) of sucrose.
   70. The aqueous pharmaceutical composition of any of embodiments 56-68, wherein the composition comprises about 3% (w/v) to about 5% (w/v) of sucrose.
   71. The aqueous pharmaceutical composition of any of embodiments 56-68, wherein the composition comprises about 4% (w/v) of sucrose.
   72. The aqueous pharmaceutical composition of any of embodiments 56-71, wherein the composition comprises about 12 mM to about 18 mM of acetate and/or pharmaceutically acceptable acetate salt.
   73. The aqueous pharmaceutical composition of any of embodiments 56-71, wherein the composition comprises about 14 mM to about 16 mM of acetate and/or pharmaceutically acceptable acetate salt.
   74. The aqueous pharmaceutical composition of any of embodiments 56-71, wherein the composition comprises about 15 mM of acetate and/or pharmaceutically acceptable acetate salt.
   75. The aqueous pharmaceutical composition of any of embodiments 56-74, wherein the composition comprises about 18 μg/mL to about 22 μg/mL of EDTA.
   76. The aqueous pharmaceutical composition of any of embodiments 56-74, wherein the composition comprises about 20 μg/mL of EDTA.
   77. The aqueous pharmaceutical composition of any of embodiments 56-76, wherein the composition comprises about 0.02% to about 0.06% of PS-20 (w/v).
   78. The aqueous pharmaceutical composition of any of embodiments 56-76, wherein the composition comprises about 0.03 to about 0.05% of PS-20 (w/v).
   79. The aqueous pharmaceutical composition of any of embodiments 56-76, wherein the composition comprises about 0.04% or about 0.06% of PS-20 (w/v).
   80. The aqueous pharmaceutical composition of any of embodiments 56-79, wherein the pH is about 4.8 to about 5.6.
   81. The aqueous pharmaceutical composition of any of embodiments 56-79, wherein the pH is about 4.9 to about 5.5.
   82. The aqueous pharmaceutical composition of any of embodiments 56-79, wherein the pH is about 5.2.
   83. The aqueous pharmaceutical composition of any of embodiments 56-82, wherein the composition comprises about 200 mg/mL of the bispecific BCMA/CD3 antibody, about 15 mM of acetate and/or pharmaceutically acceptable acetate salt, about 4% (w/v) sucrose, about 200 mM Arginine HCL, about 20 μg/mL EDTA, and about 0.04% (w/v) PS 20, and the composition has a pH of about 5.2.
   84. The aqueous pharmaceutical composition of any of embodiments 56-83, wherein the composition has a viscosity of no more than about 25 centipoise (cP) at 25° C.
   85. The aqueous pharmaceutical composition of any of embodiments 56-83, wherein the composition has a viscosity of no more than about 22 centipoise (cP) at 25° C.
   86. The aqueous pharmaceutical composition of any of embodiments 56-83, wherein the composition has a viscosity of no more than about 20 centipoise (cP) at 25° C.
   87. The aqueous pharmaceutical composition of any of embodiments 56-83, wherein the composition has a viscosity of no more than about 18 centipoise (cP) at 25° C.
   88. The aqueous pharmaceutical composition of any of embodiments 56-83, wherein the composition has a viscosity from about 10 centipoise (cP) to about 20 cP at 25° C.
   89. The aqueous pharmaceutical composition of any of embodiments 56-83, wherein the composition has a viscosity from about 15 centipoise (cP) to about 18 cP at 25° C.
   90. The aqueous pharmaceutical composition of any of embodiments 56-83, wherein the composition has a viscosity from about 16 centipoise (cP) to about 17 cP (e.g., about 16.6 cP) at 25° C.
   91. The aqueous pharmaceutical composition of any of embodiments 56-90, wherein the composition comprises high molecular weight species of the bispecific BCMA/CD3 antibody at no more than about 10% of the bispecific BCMA/CD3 antibody.
   92. The aqueous pharmaceutical composition of any of embodiments 56-90, wherein the composition comprises high molecular weight species of the bispecific BCMA/CD3 antibody at no more than about 7.5% of the bispecific BCMA/CD3 antibody.
   93. The aqueous pharmaceutical composition of any of embodiments 56-90, wherein the composition comprises high molecular weight species of the bispecific BCMA/CD3 antibody at no more than about 5% of the bispecific BCMA/CD3 antibody.
   94. The aqueous pharmaceutical composition of any of embodiments 56-93, wherein the composition has one or more of the following features:
(a) a color of solution that is colorless to ≤BY2, ≤B2, ≤Y2;
(b) an osmolality of about 612 to about 828 mOsm/kg;
(c) BMCAxCD3-mediated T-cell activation activity ranging from about 60% to about 140% relative to teclistamab reference material; and/or
(d) a main component of ≥95.0%, HMWS of ≤5.0%, and LMWS of <5.0%, as determined by SE-HPLC (or a main component of ≥90.0%, HMWS of ≤10.0%, and LMWS of <10.0%, as determined by SE-HPLC).
   95. The aqueous pharmaceutical composition of any of embodiments 56-93, wherein the composition has one or more of the following features:
(a) a color of solution that is colorless to ≤BY2, ≤B2, ≤Y2;
   (b) an osmolality of about 612 to about 828 mOsm/kg;
   (c) BMCAxCD3-mediated T-cell activation activity ranging from about 60% to about 140% relative to teclistamab reference material;
   (d) a purity of ≥95.0%, and no new peak >1.0% compared to teclistamab reference material, as determined by cSDS (reduced);
   (e) a purity of ≥90.0%, and no new peak >1.0% compared to teclistamab reference material, as determined by cSDS (non-reduced); and/or
   (f) a main component of ≥95.0%, HMWS of ≤5.0%, and LMWS of <5.0%, as determined by SE-HPLC (or a main component of ≥90.0%, HMWS of ≤10.0%, and LMWS of <10.0%, as determined by SE-HPLC).
      96. The aqueous pharmaceutical composition of any of embodiments 56-95, wherein the composition is stable.
      97. The aqueous pharmaceutical composition of embodiment 96, wherein stability of the stable composition is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.
      98. The aqueous pharmaceutical composition of embodiment 96 or embodiment 97, wherein the aqueous pharmaceutical composition is stable at a temperature of about 2-8° C. for at least two years.
      99. The aqueous pharmaceutical composition of any of embodiments 56-98, wherein the composition comprises about 180 mg/mL to about 220 mg/mL (preferably about 200 mg/mL) of the BCMAxCD3 bispecific antibody for a shelf life of at least about 6 months at 2-8° C., or for at least about 12 months at 2-8° C., or for at least about 18 months at 2-8° C., or for at least about 24 months at 2-8° C., or for at least about 30 months at 2-8° C., or for at least about 36 months at 2-8° C.
      100. The aqueous pharmaceutical composition of any of embodiments 56-99, wherein the composition is suitable for subcutaneous administration to a human subject for the treatment of a hematological cancer, such as multiple myeloma.
      101. A vial containing about 1.5 mL of the composition of embodiment 83, wherein the vial contains about 300 mg of the bispecific BCMA/CD3 antibody.
      102. A vial containing the composition of any of embodiments 56-100.
      103. The vial of embodiment 101 or embodiment 102, wherein the vial comprises a stopper pierceable by a syringe.
      104. A method of treating a hematological cancer, such as multiple myeloma, in a subject in need thereof, comprising subcutaneously administering a therapeutically effective amount of the composition of any of embodiments 56-100 to the subject.
      105. A method of treating a hematological cancer, such as multiple myeloma, in a subject in need thereof, comprising subcutaneously administering a therapeutically effective amount of the composition of any of embodiments 56-100 to the subject as a treatment dose once per week.
      106. A method of treating a hematological cancer, such as multiple myeloma, in a subject in need thereof, comprising subcutaneously administering a therapeutically effective amount of the composition of any of embodiments 56-100 to the subject as a treatment dose once per week, wherein the subject has relapsed/refractory multiple myeloma.
      107. A method of treating a hematological cancer, such as multiple myeloma, in a subject in need thereof, comprising subcutaneously administering to the subject the composition of any of embodiments 56-100 once per week after subcutaneously administering to the subject one or more step-up doses of the same bispecific BCMA/CD3 antibody contained in the composition.
      108. The aqueous pharmaceutical composition of any of embodiments 56-100 for use in the treatment of cancer.
      109. The aqueous pharmaceutical composition of any of embodiments 56-100 for use in the preparation of a medicament for the treatment of cancer.
      110. Use of the aqueous pharmaceutical composition of any of embodiments 56-100 for treating in a subject in need thereof, the use comprising administering the aqueous pharmaceutical composition to the subject in need thereof.
      111. The use of embodiment 110, wherein the administration is subcutaneous.
      112. An aqueous pharmaceutical composition comprising:
a) about 7.5 mg/mL to about 12.5 mg/mL of a bispecific B-cell mature antigen (BCMA)/cluster of differentiation 3 (CD3) antibody, the bispecific BCMA/CD3 antibody comprising:
   (1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
   (2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
   (3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
   (4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 6% (w/v) to about 10% (w/v) of sucrose;
(d) about 6 mM to about 14 mM arginine HCl;
(e) about 16 μg/mL to about 24 μg/mL of ethylenediaminetetraacetic acid (EDTA); and
(f) about 0.01% to about 0.07% polysorbate 20, wherein the composition has a pH from about 4.7 to about 5.7.
   113. The aqueous pharmaceutical composition of embodiment 112, wherein the bispecific BCMA/CD3 antibody comprises a VH1 having the amino acid sequence of SEQ ID NO:7, and a VL1 having the amino acid sequence of SEQ ID NO:8.
   114. The aqueous pharmaceutical composition of embodiment 112 or embodiment 113, wherein the bispecific BCMA/CD3 antibody comprises a VH2 having the amino acid sequence of SEQ ID NO:17, and a VL2 having the amino acid sequence of SEQ ID NO:18.
   115. The aqueous pharmaceutical composition of any of embodiments 112-114, wherein the bispecific BCMA/CD3 antibody comprises a HC1 having at least 95% sequence identity to the amino acid sequence of SEQ ID NO:9, and a LC1 having at least 95% sequence identity to the amino acid sequence of SEQ ID NO:10.
   116. The aqueous pharmaceutical composition of any of embodiments 112-114, wherein the bispecific BCMA/CD3 antibody comprises a HC1 having the amino acid sequence of SEQ ID NO:9, and a LC1 having the amino acid sequence of SEQ ID NO:10.
   117. The aqueous pharmaceutical composition of any of embodiments 112-116, wherein the bispecific BCMA/CD3 antibody comprises a HC2 having at least 95% sequence identity to the amino acid sequence of SEQ ID NO:19, and a LC2 having at least 95% sequence identity to the amino acid sequence of SEQ ID NO:20.
   118. The aqueous pharmaceutical composition of any of embodiments 112-116, wherein the bispecific BCMA/CD3 antibody comprises a HC2 having the amino acid sequence of SEQ ID NO:19, and a LC2 having the amino acid sequence of SEQ ID NO:20.
   119. The aqueous pharmaceutical composition of embodiment 112, wherein the bispecific BCMA/CD3 antibody is teclistamab.
   120. The aqueous pharmaceutical composition of any of embodiments 112-119, wherein the composition comprises about 9 mg/mL to about 11 mg/mL of the bispecific BCMA/CD3 antibody.
   121. The aqueous pharmaceutical composition of any of embodiments 112-119, wherein the composition comprises about 10 mg/mL of the bispecific BCMA/CD3 antibody.
   122. The aqueous pharmaceutical composition of any of embodiments 112-121, wherein the composition comprises about 7 mM to about 13 mM arginine HCl.
   123. The aqueous pharmaceutical composition of any of embodiments 112-121, wherein the composition comprises about 8 mM to about 12 mM arginine HCl.
   124. The aqueous pharmaceutical composition of any of embodiments 112-121, wherein the composition comprises about 10 mM arginine HCl.
   125. The aqueous pharmaceutical composition of any of embodiments 112-124, wherein the composition comprises about 6% (w/v) to about 9% (w/v) of sucrose.
   126. The aqueous pharmaceutical composition of any of embodiments 112-124, wherein the composition comprises about 7% (w/v) to about 8% (w/v) of sucrose.
   127. The aqueous pharmaceutical composition of any of embodiments 112-124, wherein the composition comprises about 7.8% (w/v) of sucrose.
   128. The aqueous pharmaceutical composition of any of embodiments 112-127, wherein the composition comprises about 12 mM to about 18 mM of acetate and/or pharmaceutically acceptable acetate salt.
   129. The aqueous pharmaceutical composition of any of embodiments 112-127, wherein the composition comprises about 14 mM to about 16 mM of acetate and/or pharmaceutically acceptable acetate salt.
   130. The aqueous pharmaceutical composition of any of embodiments 112-127, wherein the composition comprises about 15 mM of acetate and/or pharmaceutically acceptable acetate salt.
   131. The aqueous pharmaceutical composition of any of embodiments 112-130, wherein the composition comprises about 18 μg/mL to about 22 μg/mL of EDTA.
   132. The aqueous pharmaceutical composition of any of embodiments 112-130, wherein the composition comprises about 20 μg/mL of EDTA.
   133. The aqueous pharmaceutical composition of any of embodiments 112-132, wherein the composition comprises about 0.02% to about 0.06% of PS-20 (w/v).
   134. The aqueous pharmaceutical composition of any of embodiments 112-132, wherein the composition comprises about 0.03 to about 0.05% of PS-20 (w/v).
   135. The aqueous pharmaceutical composition of any of embodiments 112-132, wherein the composition comprises about 0.04% or 0.06% of PS-20 (w/v).
   136. The aqueous pharmaceutical composition of any of embodiments 112-135, wherein the pH is about 4.8 to about 5.6.
   137. The aqueous pharmaceutical composition of any of embodiments 112-135, wherein the pH is about 4.9 to about 5.5.
   138. The aqueous pharmaceutical composition of any of embodiments 112-135, wherein the pH is about 5.2.
   139. The aqueous pharmaceutical composition of any of embodiments 112-138, wherein the composition comprises about 10 mg/mL of the bispecific BCMA/CD3 antibody, about 15 mM of acetate and/or pharmaceutically acceptable acetate salt, about 7.8% (w/v) sucrose, about 10 mM Arginine HCL, about 20 μg/mL EDTA, and about 0.04% (w/v) PS 20, and the composition has a pH of about 5.2.
   140. The aqueous pharmaceutical composition of any of embodiments 112-139, wherein the composition has one or more of the following features:
(a) a color of solution that is colorless to ≤BY2, ≤B2, ≤Y2;
   (b) an osmolality of about 612 to about 828 mOsm/kg;
   (c) BMCAxCD3-mediated T-cell activation activity ranging from about 60% to about 140% relative to teclistamab reference material;
   (d) a purity of ≥95.0%, and no new peak >1.0% compared to teclistamab reference material, as determined by cSDS (reduced);
   (e) a purity of ≥90.0%, and no new peak >1.0% compared to teclistamab reference material, as determined by cSDS (non-reduced); and/or
   (f) a main component of ≥95.0%, HMWS of ≤5.0%, and LMWS of <5.0%, as determined by SE-HPLC (or a main component of ≥90.0%, HMWS of ≤10.0%, and LMWS of <10.0%, as determined by SE-HPLC).
      141. The aqueous pharmaceutical composition of any of embodiments 112-140, wherein the composition is stable.
      142. The aqueous pharmaceutical composition of embodiment 141, wherein stability of the stable composition is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.
      143. The aqueous pharmaceutical composition of embodiment 141 or embodiment 142, wherein the aqueous pharmaceutical composition is stable at a temperature of about 2-8° C. for at least two years.
      144. The aqueous pharmaceutical composition of any of embodiments 112-143, wherein the composition comprises about 8 mg/mL to about 12 mg/mL (preferably about 10 mg/mL) of the BCMAxCD3 bispecific antibody for a shelf life of at least about 6 months at 2-8° C., or for at least about 12 months at 2-8° C., or for at least about 18 months at 2-8° C., or for at least about 24 months at 2-8° C., or for at least about 30 months at 2-8° C., or for at least about 36 months at 2-8° C.
      145. The aqueous pharmaceutical composition of any of embodiments 112-144, wherein the composition is suitable for subcutaneous administration to a human subject for the treatment of a hematological cancer, such as multiple myeloma.
      146. The aqueous pharmaceutical composition of any of embodiments 112-144, wherein the composition is suitable for subcutaneous administration to a human subject as a step-dose for the treatment of a hematological cancer, such as multiple myeloma.
      147. A vial containing about 3 mL of the composition of embodiment 139, wherein the vial contains about 30 mg of the bispecific BCMA/CD3 antibody.
      148. A vial containing the composition of any of embodiments 112-146.
      149. The vial of embodiment 147 or embodiment 148, wherein the vial comprises a stopper pierceable by a syringe.
      150. A method of treating a hematological cancer, such as multiple myeloma, in a subject in need thereof, comprising:
subcutaneously administering to the subject one or more step-up doses of a composition A, wherein composition A comprises about 10 mg/mL of a bispecific BCMA/CD3 antibody, about 15 mM of acetate and/or pharmaceutically acceptable acetate salt, about 7.8% (w/v) sucrose, about 10 mM Arginine HCL, about 20 μg/mL EDTA, and about 0.04% (w/v) PS 20, wherein composition A has a pH of about 5.2;
and then subcutaneously administering to the subject a composition B once per week, wherein composition B comprises about 200 mg/mL of the bispecific BMCA/CD3 antibody, about 15 mM of acetate and/or pharmaceutically acceptable acetate salt, about 4% (w/v) sucrose, about 200 mM Arginine HCL, about 20 μg/mL EDTA, and about 0.04% (w/v) PS 20, wherein composition B has a pH of about 5.2,
wherein the bispecific BMCA/CD3 antibody comprises a HC2 having at least 95% sequence identity to the amino acid sequence of SEQ ID NO:19, and a LC2 having at least 95% sequence identity to the amino acid sequence of SEQ ID NO:20.
   151. A method of treating a hematological cancer, such as multiple myeloma, in a subject in need thereof, comprising:
subcutaneously administering to the subject one or more step-up doses of a composition A, wherein composition A comprises about 10 mg/mL of a bispecific BCMA/CD3 antibody, about 15 mM of acetate and/or pharmaceutically acceptable acetate salt, about 7.8% (w/v) sucrose, about 10 mM Arginine HCL, about 20 μg/mL EDTA, and about 0.04% (w/v) PS 20, wherein composition A has a pH of about 5.2;
and then subcutaneously administering to the subject a composition B once per week, wherein composition B comprises about 200 mg/mL of the bispecific BMCA/CD3 antibody, about 15 mM of acetate and/or pharmaceutically acceptable acetate salt, about 4% (w/v) sucrose, about 200 mM Arginine HCL, about 20 μg/mL EDTA, and about 0.04% (w/v) PS 20, wherein composition B has a pH of about 5.2,
wherein the bispecific BMCA/CD3 antibody comprises a HC2 having the amino acid sequence of SEQ ID NO:19, and a LC2 having the amino acid sequence of SEQ ID NO:20.
   152. A method of preparing the aqueous pharmaceutical composition of any of embodiments 56-100 or 112-146, the method comprising combining the bispecific BCMA/CD3 antibody with the acetate and/or pharmaceutically acceptable acetate salt, the sucrose, the arginine HCL, the EDTA, and the PS 20 to form the aqueous pharmaceutical composition.

### Description of Analytical Tests used Herein

### Analytical Tests—General Characterization

### Color of Solution

Color of solution is monitored for drug product to assess appearance and ensure it is consistent with previous batches at release and over the shelf life. Color of solution may be an indicator of product stability. To determine Color of solution, test samples are visually compared to a defined set of reference solutions.

A defined volume of liquid content is transferred into a pre-scored ampoule of same dimensions as the reference solutions. Then the content of the ampoule is visually compared to European Pharmacopoeia color reference solutions. The degree of color is determined in diffuse daylight, viewed against a white background.

### Color of Solution Material and Methods

Materials and methods are as described in European Pharmacopoeia 2.2.2,Degree of Coloration of Liquids European Pharmacopoeia (Ph. Eur.) 10th Edition monograph number 20202, July 2019. Briefly, test articles are compared against B (Brown), BY (Brownish-Yellow), and Y (Yellow) Color Reference Solution Sets.

Color of Solution results consistent with stability. In one embodiment, stability is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a color of solution of colorless to about BY4 or less, to about B4 or less, to about Y4 or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

### pH

pH Materials and Methods—A daily calibrated electronic pH meter with standardized pH electrode is used to measure the pH of test articles. All calibration solutions, reference buffers, and test articles are equilibrated to, and maintained at, 25° C. prior to and during testing.

pH results consistent with stability. In one embodiment, stability is defined as having a pH range of 4.7 to about 5.7 after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined a pH range of 4.8 to about 5.6 after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a pH range of 4.9 to about 5.5 after storage for about 12 months or more and at a temperature of about 5° C., after storage for about 12 months or more and at a temperature of about 25° C., and/or after storage for about 2 years or more and at a temperature of about 5° C.

### Turbidity

Turbidity Materials and Methods—The materials and methods are based on European Pharmacopoeia 2.2.1, Clarity and Degree of Opalescence of Liquids.

Turbidity results consistent with stability. Test results are reported in nephelometric turbidity units (NTU). In one embodiment, stability is defined as having a turbidity value of about 18 NTU or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a turbidity value of about 13 NTU or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a turbidity value of about 8 NTU or less after storage for about 12 months or more and at a temperature of about 5° C., after storage for about 12 months or more and at a temperature of about 25° C., and/or after storage for about 2 years or more and at a temperature of about 5° C.

### Analytical Tests—Particulate Matter

Particulate Matter (Sub-visible) Materials and Methods—All materials and methods are compliant with United States Pharmacopeia<788>Particulate Matter. A Compendial compliant Liquid Particle Counter instrument equipped with a compendial volume sampler set-up is used. Test particles are equilibrated to room temperature for at least 60 minutes, but no longer than 10 hours, prior to testing. Test particle vials are pooled in manner compliant with United States Pharmacopeia<788>Particulate Matter. As instructed by United States Pharmacopeia<788>Particulate Matter, four portions of pooled test article, each of appropriate volume, are removed and the number of particles equal to or greater than 10 μm and 25 μm are counted per portion. Results obtained for the first portion are disregarded and the remaining three results are used to calculate the mean number of particles for the preparation examined.

Particle Analysis (sub-vis) compendia compliant results—Testing results are to comply with United States Pharmacopoeia<788>Particulate Matter, European Pharmacopoeia 2.9.19, and Japanese Pharmacopocia XVII/6.07 Particulate Contamination: Sub-visible particles. As such, the average number of particles present in the units tested should not exceed 6000 particles per container for particles size equal to 10 μm or greater and should not exceed 600 particles per container for particles size equal to 25 μm or greater.

### Analytical Tests—Purity

### Capillary Electrophoresis Sodium Dodecyl Sulfate (cSDS)-Reduced

cSDS Reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer and certified protein molecular weight standards spanning a range of approximately 10 to 148 kDa. The instrument's ultraviolet absorption spectrophotometer detector is set at a wavelength of 220 nm and the capillary temperature is set to 25° C. For reducing sample treatment conditions, the test article (in duplicate) is mixed with SDS and 2-mercaptoethanol and then heated for a defined time and temperature to fully denature and reduce the protein. The reduced sample is injected electro-kinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected for the light chain, heavy chain, and a glycosylated heavy chain (AG HC).

cSDS Reduced results consistent with stability. In one embodiment, stability is defined as having a percent purity ≥90.0%, and no new peak >1.5% compared to a validated stock of teclistamab Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a percent purity about or more than 95.0% and no new peak more than 1.2% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a percent purity about or more than 97.0% and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

### Capillary Electrophoresis Sodium Dodecyl Sulfate (cSDS)-Non-Reduced

cSDS Non-reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer, certified protein molecular weight standards spanning a range of approximately 10 to 148 kDa, and a validated teclistamab reference material sample. The instrument's ultraviolet absorption spectrophotometer detector is set at a wavelength of 220 nm and the capillary temperature is set to 25° C. For non-reduced sample treatment conditions, the test article (in duplicate) is mixed with SDS and the alkylating reagent(N-Ethylmaleimide, to prevent disulfide bond shuffling or reformation). It is then heated for a defined time and temperature to fully denature the protein and minimize formation of fragments and artifact bands. The non-reduced sample is injected electrokinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected. The data is also analyzed for the presence of new peaks versus teclistamab reference material. Percent purity is defined as percent heavy chain+percent light chain.

cSDS Non-Reduced results consistent with stability. In one embodiment, stability is defined as having a percent purity of about 90.0% or more and no new peak more than 1.5% compared to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a percent purity of about 95.0% or more and no new peak more than 1.2% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a percent purity of about 97.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

### Size Exclusion High Performance Liquid Chromatography (SE-HPLC)

SE-HPLC Materials and Methods—Reference Material and test articles are diluted to a target protein concentration. A 20 μl volume of analyte is injected onto a 7.8 mm×30 cm size exclusion column with 5 μm particle size silica base, with a fractionation range of 10 to 500 kDa. Aqueous phosphate buffer is used as the mobile phase at a flow rate of 0.7 mL/minute and the absorbance of the eluate is monitored continuously at 280 nm. Monomer (main component or main peak), aggregates (high molecular weight species, or HMWS), and fragments (low molecular weight species, or LMWS) are separated on the column and elute at different retention times. The amounts of these species are measured by monitoring peak absorbance at 280 nm.

### SE-HPLC Results Consistent with Stability

Main Component—In one embodiment, stability is defined as having a Main Component about 90.0% or more after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Component about 95.0% or more after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Component about 97.0% after storage for about 12 months or more and at a temperature of about 5° C., after storage for about 12 months or more and at a temperature of about 25° C., and/or after storage for about 2 years or more and at a temperature of about 5° C. . . .

High Molecular Weight Species (HMWS)—In one embodiment, stability is defined as having a HMWS of about 10.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a HMWS of about 5.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a HMWS of about 3.0% or less after storage for about 12 months or more and at a temperature of about 5° C., after storage for about 12 months or more and at a temperature of about 25° C., and/or after storage for about 2 years or more and at a temperature of about 5° C.

Low Molecular Weight Species (LMWS)—In one embodiment, stability is defined as having a LMWS about 5.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a LMWS of about 2.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a LMWS about 1.0% or less after storage for about 12 months or more and at a temperature of about 5° C., after storage for about 12 months or more and at a temperature of about 25° C., and/or after storage for about 2 years or more and at a temperature of about 5° C.

### Capillary Isoelectric Focusing (cIEF)

cIEF Materials and Methods—The analytical procedure is performed on a commercially available imaging cIEF analyzer equipped with an auto sampler. Analysis employs a 100-μm inner wall-coated silica capillary with an outer wall polyimide coating. In addition, an analyte solution of dilute phosphoric acid and methylcellulose, a catholyte solution of sodium hydroxide and methylcellulose, and defined type and amount of ampholytes are used. The test articles are treated with carboxypeptidase B (CPB) to remove C-terminal lysine and eliminate ambiguities introduced by the presence of multiple C-terminal variants for each charged species. The instrument's autosampler is set to 4° C. for both pre-focusing and focusing. The Pre-focusing voltage and time are 1500 V and 1 minute respectively. The Focusing voltage and time are 3000 V and 7 minutes respectively.

### cIEF Results Consistent with Stability

Main Peak—In one embodiment, stability is defined as having a Main Peak ≤60% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Peak ≤65% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Peak ≤70% after storage for about 12 months or more and at a temperature of about 5° C., after storage for about 12 months or more and at a temperature of about 25° C., and/or after storage for about 2 years or more and at a temperature of about 5° C.

Sum of acidic peaks—In one embodiment, stability is defined as having a Sum of acidic peaks totaling ≤40% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of acidic peaks totaling ≤30% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Sum of acidic peaks totaling ≤25% after storage for about 12 months or more and at a temperature of about 5° C., after storage for about 12 months or more and at a temperature of about 25° C., and/or after storage for about 2 years or more and at a temperature of about 5° C.

Sum of basic peaks—In one embodiment, stability is defined as having a Sum of basic peaks totaling about ≤15% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of basic peaks totaling about ≤10% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Sum of basic peaks totaling about ≤8% after storage for about 12 months or more and at a temperature of about 5° C., after storage for about 12 months or more and at a temperature of about 25° C., and/or after storage for about 2 years or more and at a temperature of about 5° C.

### Analytical Tests—Quantity

### Protein Concentration by A280

Protein concentration of the drug product is determined by quantification of the absorbance at 280 nm (A280).

### Protein Concentration by A280 Materials and Methods

Measurement of protein concentration is performed using a qualified and calibrated double beam UV-Vis spectrophotometer. Test articles are diluted 1:125 using 0.9% (w/v) NaCl. Samples are measured using quartz semi-micro cuvettes (1.4 ml) with a 1 cm path length and black or frosted sides. The Spectrophotometer is set to a Wavelength of 280 nm, a slit width of 1 nm, and a response of one (1) second. 0.9% (w/v) NaCl is used as the Blank control. Protein concentration (mg/mL) is calculated by dividing the product of the Test article absorbance and dilution factor by the product of the antibody's Absorptivity Constant and instrument's path length (for example, but not limited to teclistamab's Absorptivity Constant of 1.58 (mg/mL)⁻¹ cm⁻¹ and instrument's path length of 1 cm).

### 10 mg/mL Formulation Protein Concentration Results Consistent with Stability

In one embodiment, stability is defined as having a protein concentration of 7.5 to 12.5 mg/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a protein concentration of 8 to 12 mg/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a protein concentration of 9 mg/mL to 11 mg/mL after storage for about 12 months or more and at a temperature of about 5° C., after storage for about 12 months or more and at a temperature of about 25° C., and/or after storage for about 2 years or more and at a temperature of about 5° C.

### 90 mg/mL Formulation Protein Concentration Results Consistent with Stability

In one embodiment, stability is defined as having a protein concentration of 76.5 to 103.5 mg/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a protein concentration of 85 to 95 mg/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a protein concentration of 87 mg/mL to 93 mg/mL after storage for about 12 months or more and at a temperature of about 5° C., after storage for about 12 months or more and at a temperature of about 25° C., and/or after storage for about 2 years or more and at a temperature of about 5° C.

### Analytical Tests—Potency

### Potency (BCMAxCD3 T-cell Activity)

The in vitro T-cell activation by BCMAxCD3 is demonstrated using a Nuclear factor of activated T cells-Response Element(NFAT-RE)-mediated luminescence assay

This reporter assay uses luminescence induced by activation of the NFAT (nuclear factor of activated T cells) pathway in CD3-expressing engineered effector cells as a read-out for target cell/effector cell co-engagement and is a surrogate measure of target cell killing. Daudi B lymphoblast cells, which expresses BCMA on its cell surface, are used as the target cells. The engagement of both the anti-BCMA Fab region with the BCMA expressing target cells and the anti-CD3 Fab region with the genetically engineered CD3+ T-cells are required for T-cell activation and subsequent NFAT-RE-mediated luminescence.

BCMAxCD3 T-cell Activation Materials and Methods. Qualified teclistamab is used as Reference Material and Controls. Solutions of Jurkat TCR/CD3 effector cells at 1.0×10⁶ viable cells/mL in culture media (4% Heat-Inactivated Fetal Bovine Serum in RPMI) and a solution of Daudi B lymphoblast target cells at 4×10⁵ viable cells/mL in cell culture media are prepared. Equal volumes of effector cells and target cells are aliquoted into individual wells on a 96-well cell culture plate. The plate is then incubated at 37° C. (±2° C.) with 5% (±2%) CO₂ for 16-24 hours. After incubation, Bio-GloTM Luciferase substrate is added to each well and agitated moderately on a plate shaker for at least 5 minutes. Within 30 minutes of the addition of the substate, luminescence (Relative Light Unit (RLU) values) is measured using a microtiter luminescence plate reader. Data is reported as percent bioactivity relative to Reference Material.

BCMAxCD3 T-Cell activation results consistent with stability. In one embodiment, stability is defined as 50%-150% bioactivity relative to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined 60%-140% bioactivity relative to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as ranging between about 80% to 120% bioactivity relative to Reference Material after storage for about 12 months or more and at a temperature of about 5° C., after storage for about 12 months or more and at a temperature of about 25° C., and/or after storage for about 2 years or more and at a temperature of about 5° C.

### Analytical Tests—Surfactant

### Polysorbate-20 Quantification

Polysorbate 20 is quantitatively determined by mixed-mode ion-exchange/hydrophobic HPLC.

PS 20 Materials and Methods. Analysis conducted with a gradient HPLC equipped with a 2.1×20 mm on-line column containing a 30 μm water-wetable, mixed-mode polymeric spherical sorbent particles, an ELSD, and a temperature-controlled column compartment at 30° C. The flow rate is set to 1 mL/minute and the ELSD evaporator temperature is set to 50° C. Mobile Phase A is 2% v/v Formic acid in water and Mobile Phase B is 2% v/v Formic acid in Isopropyl alcohol. Neat Polysorbate 20 is used to create calibration and check standards. Test article samples are injected neat.

Polysorbate 20 results consistent with stability. In one embodiment, stability is defined as a PS20 concentration of 0.01-0.07% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as a PS 20 concentration of 0.02-0.06% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as a PS 20 concentration of 0.03-0.05% after storage for about 12 months or more and at a temperature of about 5° C., after storage for about 12 months or more and at a temperature of about 25° C., and/or after storage for about 2 years or more and at a temperature of about 5° C.

### Analytical Tests—Routine Characterization

### Peptide Map

The purpose of this test is to measure the levels of post-translational modifications, such as oxidation, deamidation, and isomerization, that may be present in the antibody structure. Test articles are enzymatically digested to yield peptide segments. These peptides are then evaluated by Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

Peptide Mapping materials and methods. Samples are denatured with 6 M Guanidine, 50 mM Tris pH 8.0, 5 mM EDTA and filtered using 30 kDa centrifugal filter device (flow through discarded). The denatured samples are reduced with 1 M Dithiothreitol (DTT), followed by alkylation with 1 M sodium Iodoacetate, and further treated with DTT to quench the reaction. The reaction mixture is exchanged into digestion buffer (50 mM Tris pH 7.0, with 1 mM CaCl₂) via Sephadex G-25 columns with separate columns used for blanks, reference material, and test articles. An aliquot of 1 mg/mL Trypsin stock solution is added to the sample in digestion buffer yielding a 20 μL/mL trypsin concentration. The solution is incubated at 37° C. for 2 hours±30 minutes. The trypsinized solution is allowed to cool to room temperature and the enzyme is inactivated with Trifluoroacetic acid. The treated samples are evaluated by Ultra High-Performance Liquid Chromatography Mass Spectroscopy (UPLC-MS) equipped with a Waters Acquity BEH (Ethylene Bridged Hybrid) C18, 2.1×100 mm, 1.7 μm, 130 Å column and an attached auto sampler. Mobile phase A is 0.1% Formic Acid in water Mobile phase B is, 0.1% FA in acetonitrile (mobile phase B). The autosampler is set to 2-8° C., the column is set to 40° C. and the flow rate is set to 500 μL/minute. Eluted peptides are subjected to electrospray ionization and detected using a calibrated on-line mass spectrometry.

### EXAMPLES

The following examples are provided to further describe some of the embodiments disclosed herein. The examples are intended to illustrate, not to limit, the disclosed embodiments. Analytical tests described above were used to carry out experimental methods described in the examples below.

The anti-BCMA/anti-CD3 antibody teclistamab (also called Tec) (e.g., described in WO2017031104A1, the content of which is incorporated herein by reference in its entirety) was used in the examples below and was made by Janssen Pharmaceuticals. Teclistamab comprises a BCMA binding arm BCMB69 and a CD3 binding arm CD3B219, the amino acid sequences of which are shown in Table 2 and Table 3, respectively.

### Example 1: mAb Concentration Versus Viscosity Study

A study was conducted to evaluate the viscosity of BCMAxCD3 at increasing mAb concentrations. Five test formulations were evaluating ranging from 50 to 150 mg/mL mAb. The formulation for all test articles was 15 mM acetate, 8.0% (w/v) sucrose, 0.04% (w/v) polysorbate 20, and 20 μg/mL EDTA at pH 5.2. The viscosity of each test formulation was measured with a Pressure-Driven Slit viscometer.

As shown in Table 4, a positive, non-linear correlation existed between protein concentration and viscosity when formulated in 15 mM acetate, 8.0% (w/v) sucrose, 0.04% (w/v) polysorbate 20, and 20 μg/mL EDTA at pH 5.2. Further, the viscosity values observed at high mAb concentration may be unsuitable for subcutaneous administration.

TABLE 4
Study Test Formulations

| mAb concentration (mg/mL) | Viscosity (cP) |
|---|---|
| 50 | 2.6 |
| 75 | 6.2 |
| 100 | 14.1 |
| 125 | 39.7 |
| 150 | 67.2 |

### Example 2: Effect of pH and Excipient Species on Viscosity of High Concentration BCMAxCD3 Formulations

A screening viscosity study was conducted to evaluate twelve high protein concentration formulations with varied excipient species and pH values (see Table X below). All test formulation contained 180 mg/mL BCMAxCD3 and 8% (w/v) sucrose. Test formulation varied by buffer species (acetate or histidine at 10 mM), pH (5.2, 5.6, 6.4) and excipient species (none, Proline, Arginine-HCl, NaCl at 100 mM). The viscosity of each test formulation was measured with a Pressure-Driven Slit viscometer.

Study results are shown table 5 below. The data shows a positive correlation between increased pH and increased viscosity regardless of the presence of species excipient in the formulation. The addition of Arg-HCl or NaCl reduced viscosity at all pH values versus no excipient with Arg-HCl consistently showing the most potent effect. While Proline modestly reduced viscosity at pH 5.6, it also modestly increased viscosity at pH 5.2 and dramatically increase viscosity at pH 6.4 versus corresponding test formulations that did not contain excipient. Taken together, the results of the study demonstrate that high concentration BCMAxCD3 formulations at pH 5.2 and containing Arg-HCl showed the greatest reduction of viscosity.

TABLE 5
Viscosity of Test Formulations

| Form- | BCMA × CD3 | Sucrose | Buffer | | Excipient | Viscosity |
|---|---|---|---|---|---|---|
| ulation | (mg/mL) | (% (w/v)) | (at 10 mM) | pH | (at 100 mM) | (cP) |
| 1 | 130 | 8 | Acetate | 5.2 | none | 26.0 |
| 2 | | | | | Proline | 28.2 |
| 3 | | | | | Arg-HCl | 9.3 |
| 4 | | | | | NaCl | 12.4 |
| 5 | | | Histidine | 5.6 | none | 45.5 |
| 6 | | | | | Proline | 30.3 |
| 7 | | | | | Arg-HCl | 12.6 |
| 8 | | | | | NaCl | 13.6 |
| 9 | | | | 6.4 | none | 94.7 |
| 10 | | | | | Proline | 153.2 |
| 11 | | | | | Arg-HCl | 14.4 |
| 12 | | | | | NaCl | 39.2 |

### Example 3—Effect of Arg-HCl Concentration on the Viscosity of High Concentration BCMAxCD3 Formulations

A study was conducted to evaluate the effect of Arg-HCl concentration on the viscosity of high concentration BCMAxCD3 formulations. Formulations were evaluating at 120 mg/mL and 150 mg/mL BCMAxCD3. Each protein concentration was evaluated from 25 to 200 mM Arg-HCl and a control formulation containing no ArgHCl. The remaining formulation components for all test articles were 15 mM acetate, 8.0% (w/v) sucrose, 0.04% (w/v) polysorbate 20, and 20 μg/mL EDTA at pH 5.2. The viscosity of each test formulation was measured with a Pressure-Driven Slit viscometer.

As shown in Table 6, a negative correlation exists between Arg-HCl conc concentration and viscosity when formulated in 15 mM acetate, 8.0% (w/v) sucrose, 0.04% (w/v) polysorbate 20, and 20 μg/mL EDTA at pH 5.2. This trend is observed in both the 120 mg/ml and 150 mg/mL BCMAxCD3 formulation with, consistent with previous data, the 120 mg/mL formulation exhibiting overall lower viscosity values than the 150 mg/mL formulation.

TABLE 6
Viscosity (cP) of Arg-HCL containing
high concentration mAb formulations

| BCMA × CD3 | | |
|---|---|---|
| concentration (mg/mL) | Arginine-HCl (mM) | Viscosity (cP) |
| 120 | 0 | 19.5 |
| | 25 | 7.0 |
| | 50 | 6.2 |
| | 100 | 5.3 |
| | 200 | 4.7 |
| 150 | 0 | 63.3 |
| | 25 | 14.0 |
| | 50 | 11.9 |
| | 100 | 9.5 |
| | 200 | 7.7 |

### Example 4—Effect of Arg-HCl and sucrose concentration on the viscosity of high concentration BCMAxCD3 formulations

A study was conducted to evaluate the effect of Arg-HCl and sucrose concentration on the viscosity of high concentration BCMAxCD3 formulations. Test formulations were evaluated from 100 to 400 mM Arg-HCl at either 4% or 8% (w/v) sucrose. The remaining formulation components for all test articles were 200 mg/mL mAb, 15 mM acetate, 0.04% (w/v) polysorbate 20, and 20 μg/mL EDTA at pH 5.2. The viscosity of each test formulation was measured with a Pressure-Driven Slit viscometer.

As shown in table 7, within a given Arg-HCl concentration, formulations containing 4% (w/v) sucrose consistently exhibited lower viscosity values than corresponding 8% (w/v) formulations when formulated at 200 mg/mL mAb in 15 mM acetate, 0.04% (w/v) polysorbate 20, and 20 μg/mL EDTA at pH 5.2. Also when formulated at 200 mg/mL, and regardless of sucrose concentration, a general negative correlation was seen between increased Arg-HCL concentration and viscosity. However, the viscosity values at 400 mM Arg-HCl were not sustainably different than those at 200 mM Arg-HCl, with the viscosity value of the 400 mM Arg-HCl 4% (w/v) sucrose test formulation being slightly higher than the 200 mM Arg-HCl 4% (w/v) sucrose. Thus, at 200 mg/mL mAb and between 4-8% (w/v) sucrose, the negative correlation between Arg-HCL concentration and viscosity appeared to plateau at 200 mM Arg-HCl.

TABLE 7
Viscosity (cP) of high concentration mAb formulations
at varied Arg-HCL and sucrose concentrations

| Test Formulation | Arg-HCl (mM) | Sucrose (% w/v) | Viscosity (cP) |
|---|---|---|---|
| 1 | 100 | 4 | 26.2 |
| 2 | | 8 | 34.0 |
| 3 | 200 | 4 | 19.1 |
| 4 | | 8 | 25.3 |
| 5 | 400 | 4 | 19.7 |
| 6 | | 8 | 22.2 |

### Example 5—Effect of Surfactant Species on the Viscosity of High Concentration BCMAxCD3 Formulations

A study was conducted to evaluate the effect of surfactant species on the viscosity of high concentration BCMAxCD3 formulations. P188 was evaluated at 0.5% and 0.05% (w/v). PS80 and PS20 were each evaluated at 0.04% (w/v). The remaining formulation components for all test articles were 200 mg/ml mAb, 15 mM acetate, 4% (w/v) sucrose, and 20 μg/mL EDTA at pH 5.2. The viscosity of each test formulation was measured with a Pressure-Driven Slit viscometer.

As shown in table 8, P188 Test Formulations had a somewhat higher viscosity value that polysorbate containing Test Formulations with 0.5% (w/v) P188 exhibiting a slightly higher viscosity value than 0.05% (w/v) P188. The viscosity values for the polysorbate containing Test Articles were nearly identical. Thus, at 200 mg/mL mAb, 15 mM acetate, 4% (w/v) sucrose, and 20 μg/mL EDTA at pH 5.2, polysorbate species had no practical impact on viscosity.

TABLE 8
Effect of surfactant species on viscosity
(cP) of high concentration mAb formulations

| | Surfactant | Surfactant | |
|---|---|---|---|
| Test Formulation | Species | Conc. (w/v) | Viscosity (cP) |
| 1 | P188 | 0.50% | 23.5 |
| 2 | P188 | 0.05% | 22.7 |
| 3 | PS80 | 0.04% | 21.4 |
| 4 | PS20 | 0.04% | 21.2 |

### Example 6: 200 mg/mL Drug Product Formulation: Composition and Components of Primary Container

Provided herein is a tabular summary of the composition of an exemplary 200 mg/mL Teclistamab Drug Product Formulation in table 9.

TABLE 9
Composition of 200 mg/mL Teclistamab Drug Product

| Component | Amount per mL | |
|---|---|---|
| Teclistamab | 200 | mg |
| Sodium acetate trihydrate | 1.497 | mg |
| Glacial acetic acid | 0.240 | mg |
| Sucrose | 4 | mg |
| Arginine-HCL | 42.1 | mg |
| Polysorbate 20 | 0.40 | mg |
| EDTA Disodium salt, Dihydrate | 0.02 | mg |

| | |
|---|---|
| Water for Injection | q.s to 1.079 g |

The 200 mg/mL Teclistamab drug product(DP) primary packaging consists of a glass vial, a polymer vial stopper, and an aluminum seal. Table 9A lists the specific components for the primary packaging material of the 200 mg/mL DP presentation.

TABLE 9A
300 mg DP Primary packaging material components

| Component | Description |
|---|---|
| Glass vial | 2 mL glass Type 1 borosilicate |
| Stopper | 13 mm Stopper with Flurotec Coating |
| Seals | 13 mm silver aluminum seal with flip-off cap |

### Example 7: 200 mg/mL Stability Study

This study was conducted to monitor high concentration BCMAxCD3 Drug Product attributes placed on stability under various environmental conditions and lengths of time.

BCMAxCD3 DP was formulated at 200 mg/mL in 15 mM acetate, 4% sucrose (w/v), 200 mM ArgHCl, 20 μg/mL EDTA, 0.04% PS 20 (w/v), at pH 5.2.

Study test articles were prepared by aliquoting the formulated DP into 2R vials at a fill volume of 1.5 mL. The vials were stoppered, capped, and crimp sealed.

All studies were to be performed with vials in an inverted orientation. Study parameters are shown in table 10.

TABLE 10
Study parameters

| Stability Classification | Storage condition | Duration (Months) |
|---|---|---|
| Real-time | 5 ± 3° C. | 36 |
| Accelerated | 25 ± 2° C./60% RH | 12 |
| Stressed | 40 ± 2° C./75% RH | 6 |

### Stability Study Results

The stability results for teclistamab DP held under recommended, accelerated, and stressed conditions are listed below. Results observed for teclistamab DP held at recommended storage conditions are consistent with a stable pharmaceutical composition.

Results for teclistamab DP held at accelerated and stressed conditions showed the rates of degradation for Drug Product exposed to prolonged accelerated and stressed storage conditions Of particular note, results observed for teclistamab DP held at accelerated conditions (25° C.) are similar results observed for teclistamab DP held at recommended storage conditions (2-8° C.)

TABLE 11
Stability Results for 200 mg/mL BCMA Drug Product Stored at 5° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | Color of | | Turbidity | particles | particles | Purity: | |
| Months | Solution | pH | (NTU) | per vial | per vial | % | new peaks |
| 0 | ≤B5, ≤BY4, | 5.2 | 17 | 19 | 11 | 99.2 | No new peak > 1.0% |
| | <Y4 | | | | | | compared to Reference |
| | | | | | | | Material |
| 3 | ≤B5, ≤BY6, | 5.3 | 21 | 4 | 0 | 98.9 | No new peak > 1.0% |
| | ≤Y6 | | | | | | compared to Reference |
| | | | | | | | Material |
| 6 | | | | | | | No new peak > 1.0% |
| | | | | | | | compared to Reference |
| | | | | | | | Material |
| 9 | | | | | | | No new peak > 1.0% |
| | | | | | | | compared to Reference |
| | | | | | | | Material |
| 12 | | | | | | | No new peak > 1.0% |
| | | | | | | | compared to Reference |
| | | | | | | | Material |
| 18 | | | | | | | |
| 24 | | | | | | | |
| 36 | | | | | | | |

TABLE 12
Stability Results for 200 mg/mL BCMA Drug Product Stored at 5° C. (Continued)

| | | | SE HPLC | | | | |
|---|---|---|---|---|---|---|---|
| | cSDS (Non-reduced) | | Main | | | Protein Conc. | Potency T-Cell |
| | Purity | | Component | HMWS | LMWS | by A₂₈₀ | Activation Assay |
| Months | (%) | New Peaks (%) | (%) | (%) | (%) | (mg/mL) | % of RM activity |
| 0 | 98.6 | No new peak > 1.0% | 99.0 | 1.0 | <0.1 | 202 | 100 |
| | | compared to | | | | | |
| | | Reference Material | | | | | |
| 3 | 98.3 | No new peak > 1.0% | 99.0 | 1.0 | <0.1 | 200 | 96 |
| | | compared to | | | | | |
| | | Reference Material | | | | | |
| 6 | | No new peak > 1.0% | | | | | |
| | | compared to | | | | | |
| | | Reference Material | | | | | |
| 9 | | No new peak > 1.0% | | | | | |
| | | compared to | | | | | |
| | | Reference Material | | | | | |
| 12 | | No new peak > 1.0% | | | | | |
| | | compared to | | | | | |
| | | Reference Material | | | | | |
| 18 | | | | | | | |
| 24 | | | | | | | |
| 36 | | | | | | | |

TABLE 13
Stability Results for 200 mg/mL BCMA Drug
Product Stored at 5° C. (Continued)

| | IE-HPLC | | | |
|---|---|---|---|---|
| | Main | Sum of acidic | Sum of Basic | Polysorbate 20 |
| Months | peak (%) | peaks (%) | peaks (%) | (%) |
| 0 | 79 | 17 | 3 | 0.03 |
| 3 | 80 | 17 | 3 | 0.03 |
| 9 | | | | |
| 12 | | | | |
| 18 | | | | |
| 24 | | | | |
| 36 | | | | |

TABLE 14
Stability Results for 200 mg/mL BCMA Drug
Product Stored at 5° C. (Continued)

| | Post Translational Modification | | |
|---|---|---|---|
| | BCMA HC | CD3 HC | |
| | Met253/CD3 HC | Asn103/Asn106 | BCMA HC Asp101 |
| | Met257 Oxidation | Deamidation | Isomerization |
| Months | (%) | (%) | (%) |
| 0 | 3.6 | 9.4 | 3.1 |
| 3 | | | |
| 6 | | | |
| 9 | | | |
| 12 | | | |
| 18 | | | |
| 24 | | | |
| 36 | | | |

TABLE 15
Stability Results for 200 mg/mL BCMA Drug Product Stored at 25° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | Color of | | Turbidity | particles | particles | Purity: | |
| Months | Solution | pH | (NTU) | per vial | per vial | % | new peaks |
| 0 | ≤B5, ≤BY4, | 5.2 | 17 | 19 | 11 | 99.2 | No new peak > 1.0% |
| | ≤Y4 | | | | | | compared to Reference |
| | | | | | | | Material |
| 1 | ≤B5, ≤BY4, | 5.3 | 15 | 154 | 13 | 98.4 | No new peak > 1.0% |
| | ≤Y4 | | | | | | compared to Reference |
| | | | | | | | Material |
| 3 | | | | | | | |
| 6 | | | | | | | |
| 9 | | | | | | | |
| 12 | | | | | | | |

TABLE 16
Stability Results for 200 mg/mL BCMA Drug Product Stored at 25° C. (Continued)

| | | | SE HPLC | | | | |
|---|---|---|---|---|---|---|---|
| | cSDS (Non-reduced) | | Main | | | Protein Conc. | Potency T-Cell |
| | Purity | | Component | HMWS | LMWS | by A₂₈₀ | Activation Assay |
| Months | (%) | New Peaks (%) | (%) | (%) | (%) | (mg/mL) | % of RM activity |
| 0 | 98.6 | No new peak > 1.0% | 99 | 1.0 | <0.1 | 202 | 100 |
| | | compared to | | | | | |
| | | Reference Material | | | | | |
| 1 | 98.5 | No new peak > 1.0% | 98.7 | 1.3 | <0.1 | 201 | 85 |
| | | compared to | | | | | |
| | | Reference Material | | | | | |
| 3 | | | | | | | |
| 6 | | | | | | | |
| 9 | | | | | | | |
| 12 | | | | | | | |

TABLE 17
Stability Results for 200 mg/mL BCMA Drug
Product Stored at 25° C. (Continued)

| | IE-HPLC | | | |
|---|---|---|---|---|
| | Main | Sum of acidic | Sum of Basic | Polysorbate 20 |
| Months | peak (%) | peaks (%) | peaks (%) | (%) |
| 0 | 79 | 17 | 3 | 0.03 |
| 1 | 78 | 18 | 4 | 0.03 |
| 3 | | | | |
| 6 | | | | |
| 9 | | | | |
| 12 | | | | |

TABLE 18
Stability Results for 200 mg/mL BCMA Drug
Product Stored at 25° C. (Continued)

| | Post Translational Modification | | |
|---|---|---|---|
| | BCMA HC | CD3 HC | |
| | Met253/CD3 HC | Asn103/Asn106 | BCMA HC Asp101 |
| | Met257 Oxidation | Deamidation | Isomerization |
| Months | (%) | (%) | (%) |
| 0 | 3.6 | 9.4 | 3.1 |
| 3 | | | |
| 6 | | | |
| 9 | | | |
| 12 | | | |

TABLE 19
Stability Results for 200 mg/mL BCMA Drug Product Stored at 40° C.

| | | | | Particulate Matter | | | |
|---|---|---|---|---|---|---|---|
| | | | | Sub-visible | | | |
| | | | | ≥10 μm: | ≥25 μm: | cSDS (Reduced) | |
| | | | Turbidity | particles | particles | Purity: | |
| Months | Color of Solution | pH | (NTU) | per vial | per vial | % | new peaks |
| 0 | <B5, ≤BY4, | 5.2 | 17 | 19 | 11 | 99.2 | No new peak > 1.0% |
| | <Y4 | | | | | | compared to |
| | | | | | | | Reference Material |
| 1 | ≤B5, ≤BY4, | 5.3 | 37 | 162 | 125 | 94.5 | No new peak > 1.0% |
| | ≤Y4 | | | | | | compared to |
| | | | | | | | Reference Material |
| 3 | | | | | | | |
| 6 | | | | | | | |

TABLE 20
Stability Results for 200 mg/mL BCMA Drug Product Stored at 40° C. (Continued)

| | | | SE HPLC | | | | |
|---|---|---|---|---|---|---|---|
| | cSDS (Non-reduced) | | Main | | | Protein Conc. | Potency T-Cell |
| | Purity | | Component | HMWS | LMWS | by A₂₈₀ | Activation Assay |
| Months | (%) | New Peaks (%) | (%) | (%) | (%) | (mg/mL) | % of RM activity |
| 0 | 98.6 | No new peak > 1.0% | 99 | 1.0 | <0.1 | 202 | 100 |
| | | compared to | | | | | |
| | | Reference Material | | | | | |
| 1 | 94.6 | No new peak > 1.0% | 94.5 | 5.1 | 0.4 | 205 | 49 |
| | | compared to | | | | | |
| | | Reference Material | | | | | |
| 3 | | | | | | | |
| 6 | | | | | | | |

TABLE 21
Stability Results for 200 mg/mL BCMA Drug
Product Stored at 40° C. (Continued)

| | IE-HPLC | | | |
|---|---|---|---|---|
| | Main | Sum of acidic | Sum of Basic | Polysorbate 20 |
| Months | peak (%) | peaks (%) | peaks (%) | (%) |
| 0 | 79 | 17 | 3 | 0.03 |
| 1 | 62 | 24 | 14 | 0.03 |
| 3 | | | | |
| 6 | | | | |

TABLE 22
Stability Results for 200 mg/mL BCMA Drug
Product Stored at 40° C. (Continued)

| | Post Translational Modification | | |
|---|---|---|---|
| | BCMA HC | CD3 HC | |
| | Met253/CD3 HC | Asn103/Asn106 | BCMA HC Asp101 |
| | Met257 Oxidation | Deamidation | Isomerization |
| Months | (%) | (%) | (%) |
| 0 | 3.6 | 9.4 | 3.1 |
| 1 | | | |
| 3 | | | |
| 6 | | | |

1. An aqueous pharmaceutical composition comprising:
a) about 150 mg/mL to about 250 mg/mL of a bispecific B-cell mature antigen (BCMA)/cluster of differentiation 3 (CD3) antibody, the bispecific BCMA/CD3 antibody comprising:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively; and
b) arginine HCl,
wherein the composition has a viscosity of no more than about 25 centipoise (cP) at 25° C.

2. The aqueous pharmaceutical composition of claim 1, wherein the bispecific BCMA/CD3 antibody comprises a VH1 having the amino acid sequence of SEQ ID NO:7, and a VL1 having the amino acid sequence of SEQ ID NO:8.

3. The aqueous pharmaceutical composition of claim 1, wherein the bispecific BCMA/CD3 antibody comprises a VH2 having the amino acid sequence of SEQ ID NO:17, and a VL2 having the amino acid sequence of SEQ ID NO:18.

4. The aqueous pharmaceutical composition of claim 1, wherein the bispecific BCMA/CD3 antibody comprises a HC1 having at least 95% sequence identity to the amino acid sequence of SEQ ID NO:9, and a LC1 having at least 95% sequence identity to the amino acid sequence of SEQ ID NO:10.

5. The aqueous pharmaceutical composition of claim 1, wherein the bispecific BCMA/CD3 antibody comprises a HC1 having the amino acid sequence of SEQ ID NO:9, and a LC1 having the amino acid sequence of SEQ ID NO:10.

6. The aqueous pharmaceutical composition of claim 1, wherein the bispecific BCMA/CD3 antibody comprises a HC2 having at least 95% sequence identity to the amino acid sequence of SEQ ID NO:19, and a LC2 having at least 95% sequence identity to the amino acid sequence of SEQ ID NO:20.

7. The aqueous pharmaceutical composition of claim 1, wherein the bispecific BCMA/CD3 antibody comprises a HC2 having the amino acid sequence of SEQ ID NO:19, and a LC2 having the amino acid sequence of SEQ ID NO:20.

8. The aqueous pharmaceutical composition of claim 1, wherein the bispecific BCMA/CD3 antibody is teclistamab.

9. The aqueous pharmaceutical composition of claim 1, wherein the composition comprises about 180 mg/mL to about 220 mg/mL of the bispecific BCMA/CD3 antibody.

10. The aqueous pharmaceutical composition of claim 1, wherein the composition comprises about 200 mg/mL of the bispecific BCMA/CD3 antibody.

11. The aqueous pharmaceutical composition of claim 1, wherein the composition comprises about 100 mM to about 300 mM arginine HCl.

12. The aqueous pharmaceutical composition of claim 1, wherein the composition comprises about 150 mM to about 250 mM arginine HCl.

13. The aqueous pharmaceutical composition of claim 1, wherein the composition comprises about 200 mM arginine HCl.

14. The aqueous pharmaceutical composition of claim 1, wherein the composition has a viscosity of no more than about 22 centipoise (cP) at 25° C.

15. The aqueous pharmaceutical composition of claim 1, wherein the composition has a viscosity of no more than about 20 centipoise (cP) at 25° C.

16. The aqueous pharmaceutical composition of claim 1, wherein the composition has a viscosity of no more than about 18 centipoise (cP) at 25° C.

17. The aqueous pharmaceutical composition of claim 1, wherein the composition has a viscosity from about 10 centipoise (cP) to about 20 cP at 25° C.

18. The aqueous pharmaceutical composition of claim 1, wherein the composition has a viscosity from about 15 centipoise (cP) to about 18 cP at 25° C.

19. The aqueous pharmaceutical composition of claim 1, wherein the composition has a viscosity from about 16 centipoise (cP) to about 17 cP at 25° C.

20. The aqueous pharmaceutical composition of claim 1, wherein the composition comprises high molecular weight species of the bispecific BCMA/CD3 antibody at no more than about 10% of the bispecific BCMA/CD3 antibody.

21. The aqueous pharmaceutical composition of claim 1, wherein the composition comprises high molecular weight species of the bispecific BCMA/CD3 antibody at no more than about 7.5% of the bispecific BCMA/CD3 antibody.

22. The aqueous pharmaceutical composition of claim 1, wherein the composition comprises high molecular weight species of the bispecific BCMA/CD3 antibody at no more than about 5% of the bispecific BCMA/CD3 antibody.

23. The aqueous pharmaceutical composition of claim 1, wherein the composition has one or more of the following features:
(a) a color of solution that is colorless to ≤BY2, ≤B2, ≤Y2;
(b) an osmolality of about 612 to about 828 mOsm/kg;
(c) BMCAxCD3-mediated T-cell activation activity ranging from about 60% to about 140% relative to teclistamab reference material; and/or
(d) a main component of ≥95.0%, HMWS of ≤5.0%, and LMWS of <5.0%, as determined by SE-HPLC (or a main component of ≥90.0%, HMWS of ≤10.0%, and LMWS of <10.0%, as determined by SE-HPLC).

24. The aqueous pharmaceutical composition of claim 1, wherein the composition has one or more of the following features:
(a) a color of solution that is colorless to ≤BY2, ≤B2, ≤Y2;
(b) an osmolality of about 612 to about 828 mOsm/kg;
(c) BMCAxCD3-mediated T-cell activation activity ranging from about 60% to about 140% relative to teclistamab reference material;
(d) a purity of ≥95.0%, and no new peak >1.0% compared to teclistamab reference material, as determined by cSDS (reduced);
(e) a purity of ≥90.0%, and no new peak >1.0% compared to teclistamab reference material, as determined by cSDS (non-reduced); and/or
(f) a main component of ≥95.0%, HMWS of ≤5.0%, and LMWS of <5.0%, as determined by SE-HPLC (or a main component of ≥90.0%, HMWS of ≤10.0%, and LMWS of <10.0%, as determined by SE-HPLC).

25. The aqueous pharmaceutical composition of claim 1, wherein the composition is stable.

26. The aqueous pharmaceutical composition of claim 25, wherein stability of the stable composition is defined based on color of solution, pH, turbidity, percentage of purity, percentage of new peaks, percentage of main component, percentage of high molecular weight species (HWMS), percentage of low molecular weight species (LMWS), percentage of sum of acidic peaks, percentage of sum of basic peaks, protein concentration, percentage of T cell activation, percentage of PS 20 (w/v), or any combination thereof.

27. The aqueous pharmaceutical composition of claim 25, wherein the aqueous pharmaceutical composition is stable at a temperature of about 2-8° C. for at least two years.

28. The aqueous pharmaceutical composition of claim 1, wherein the composition comprises about 180 mg/mL to about 220 mg/ml of the BCMAxCD3 bispecific antibody for a shelf life of at least about 6 months at 2-8° C., or for at least about 12 months at 2-8° C., or for at least about 18 months at 2-8° C., or for at least about 24 months at 2-8° C., or for at least about 30 months at 2-8° C., or for at least about 36 months at 2-8° C.

29. The aqueous pharmaceutical composition of claim 1, wherein the composition is suitable for subcutaneous administration to a human subject for the treatment of a hematological cancer, such as multiple myeloma.

30. The aqueous pharmaceutical composition of claim 1, wherein the composition comprises about 2% (w/v) to about 6% (w/v) of sucrose.

31. The aqueous pharmaceutical composition of claim 1, wherein the composition comprises about 3% (w/v) to about 5% (w/v) of sucrose.

32. The aqueous pharmaceutical composition of claim 1, wherein the composition comprises about 4% (w/v) of sucrose.

33. The aqueous pharmaceutical composition of claim 1, wherein the composition comprises about 12 mM to about 18 mM of acetate and/or pharmaceutically acceptable acetate salt.

34. The aqueous pharmaceutical composition of claim 1, wherein the composition comprises about 14 mM to about 16 mM of acetate and/or pharmaceutically acceptable acetate salt.

35. The aqueous pharmaceutical composition of claim 1, wherein the composition comprises about 15 mM of acetate and/or pharmaceutically acceptable acetate salt.

36. The aqueous pharmaceutical composition of claim 1, wherein the composition comprises about 18 μg/mL to about 22 μg/mL of EDTA.

37. The aqueous pharmaceutical composition of claim 1, wherein the composition comprises about 20 μg/mL of EDTA.

38. The aqueous pharmaceutical composition of claim 1, wherein the composition comprises about 0.02% to about 0.06% of PS-20 (w/v).

39. The aqueous pharmaceutical composition of claim 1, wherein the composition comprises about 0.03 to about 0.05% of PS-20 (w/v).

40. The aqueous pharmaceutical composition of claim 1, wherein the composition comprises about 0.04% of PS-20 (w/v).

41. The aqueous pharmaceutical composition of claim 1, wherein the pH of the composition is about 4.8 to about 5.6.

42. The aqueous pharmaceutical composition of claim 1, wherein the pH of the composition is about 4.9 to about 5.5.

43. The aqueous pharmaceutical composition of claim 1, wherein the pH of the composition is about 5.2.

44. The aqueous pharmaceutical composition of claim 1, wherein the composition comprises about 200 mg/mL of the bispecific BCMA/CD3 antibody, about 15 mM of acetate and/or pharmaceutically acceptable acetate salt, about 4% (w/v) sucrose, about 200 mM Arginine HCL, about 20 μg/mL EDTA, and about 0.04% (w/v) PS 20, and the composition has a pH of about 5.2.

45. A vial containing about 1.5 mL of the composition of claim 44, wherein the vial contains about 300 mg of the bispecific BCMA/CD3 antibody.

46. A vial containing the composition of claim 1.

47. The vial of claim 45, wherein the vial comprises a stopper pierceable by a syringe.

48. A method of treating a hematological cancer, such as multiple myeloma, in a subject in need thereof, comprising subcutaneously administering a therapeutically effective amount of the composition of claim 1 to the subject.

49. A method of treating a hematological cancer, such as multiple myeloma, in a subject in need thereof, comprising subcutaneously administering a therapeutically effective amount of the composition of claim 1 to the subject as a treatment dose once per week.

50. A method of treating a hematological cancer, such as multiple myeloma, in a subject in need thereof, comprising subcutaneously administering a therapeutically effective amount of the composition of claim 1 to the subject as a treatment dose once per week, wherein the subject has relapsed/refractory multiple myeloma.

51. A method of treating a hematological cancer, such as multiple myeloma, in a subject in need thereof, comprising subcutaneously administering to the subject the composition of claim 1 once per week after subcutaneously administering to the subject one or more step-up doses of the same bispecific BCMA/CD3 antibody contained in the composition.

52.-55. (canceled)

56. An aqueous pharmaceutical composition comprising:
a) about 150 mg/mL to about 250 mg/mL of a bispecific B-cell mature antigen (BCMA)/cluster of differentiation 3 (CD3) antibody, the bispecific BCMA/CD3 antibody comprising:
(1) a first heavy chain (HC1) comprising a HC1 variable region 1 (VH1), wherein the VH1 comprises a heavy chain complementarity determining region 1 (HCDR1), a HCDR2, and a HCDR3 having the amino acid sequences of SEQ ID NOs:1, 2, and 3, respectively;
(2) a first light chain (LC1) comprising a LC1 variable region (VL1), wherein the VL1 comprises a light chain complementarity determining region 1 (LCDR1), a LCDR2, and a LCDR3 having the amino acid sequences of SEQ ID NOs:4, 5, and 6, respectively;
(3) a second heavy chain (HC2) comprising a HC2 variable region 2 (VH2), wherein the VH2 comprises a heavy chain complementarity determining region 1 (HCDR1), a HCDR2, and a HCDR3 having the amino acid sequences of SEQ ID NOs:11, 12, and 13, respectively; and
(4) a second light chain (LC2) comprising a LC2 variable region 2 (VL2), wherein the VL2 comprises a light chain complementarity determining region 1 (LCDR1), a LCDR2, and a LCDR3 having the amino acid sequences of SEQ ID NOs:14, 15, and 16, respectively;
(b) about 10 mM to about 20 mM of acetate and/or pharmaceutically acceptable acetate salt;
(c) about 1% (w/v) to about 7% (w/v) of sucrose;
(d) about 100 mM to about 300 mM arginine HCl;
(e) about 16 μg/mL to about 24 μg/mL of ethylenediaminetetraacetic acid (EDTA); and
(f) about 0.01% to about 0.07% polysorbate 20 (w/v), wherein the composition has a pH from about 4.7 to about 5.7.

57.-111. (canceled)
